(19) United States
(12) Patent Application Publication
(10) Pub. No.: US 2009/0048193 A1
(43) Pub. Date: Feb. 19, 2009

(54) Administration of the REG1 anticoagulation system

(76) Inventors: Christopher P. Rusconi, Durham, NC (US); Ross M. Tonkens, Cary, NC (US)

Correspondence Address: KING & SPALDING LLP, 1180 PEACHTREE STREET, ATLANTA, GA, 30309-3521, US

(21) Appl. No.: 11/805,950

(22) Filed: May 25, 2007

Related U.S. Application Data
(60) Provisional application No. 60/808,987, filed on May 26, 2006.
(60) Provisional application No. 60/847,809, filed on Sep. 27, 2006.
(60) Provisional application No. 60/865,352, filed on Nov. 10, 2006.

(51) Int. Cl. A61K 31/7088

(52) U.S. Cl. 514/44

(57) ABSTRACT

An improved method of administration of an aptamer and antidote system to regulate blood coagulation in a host is provided based on weight adjusted or body mass index-adjusted dosing of the components of the system to provide a desired pharmacodynamic response. In addition, a method of reversing activity of the aptamer to a desired extent is provided where an antidote dose is based solely on its relationship to the aptamer dose.

### CROSS-REFERENCE TO RELATED APPLICATIONS

This application claims priority to U.S. Provisional Application No. 60/808,987, filed May 26, 2006, U.S. Provisional Application No. 60/847,809, filed Sep. 27, 2006 and U.S. Provisional Application No. 60/865,352, filed Nov. 10, 2006, all entitled “Administration of the REG1 Anticoagulation System,” the disclosures of which are incorporated herein in their entirety.

### FIELD OF THE INVENTION

An improved method of administration of an aptamer and antidote system to regulate blood coagulation in a host is provided based on weight adjusted or body mass index-adjusted dosing of the components of the system.

### BACKGROUND

### Acute Care Anticoagulation

Given the central role of thrombosis in the pathobiology of acute ischemic heart disease, injectable anticoagulants have become the foundation of medical treatment for patients presenting with acute coronary syndromes, such as unstable angina, and myocardial infarction and for those undergoing coronary revascularization procedures (Harrington et al., 2004; Popma et al., 2004). Currently available anticoagulants include unfractionated heparin (UFH), the low molecular weight heparins (LMWH), and the direct thrombin inhibitors (DTI) such as recombinant hirudin, bivalirudin, and argatroban. The present paradigm both for anticoagulant use and for continued antithrombotic drug development is to establish a balance between efficacy, which means reducing the risk of ischemic events, and safety, which means minimizing the risk of bleeding (Harrington et al., 2004). Each of the available agents carries an increased risk of bleeding relative to placebo.

The major adverse event associated with anticoagulant and antithrombotic drugs is bleeding, which can cause permanent disability and death (Ebbesen et al., 2001; Levine et al., 2004). Generally, cardiovascular clinicians have been willing to trade off an increased risk of bleeding when a drug can reduce the ischemic complications of either the acute coronary syndromes or of coronary revascularization procedures. However, recent data have suggested that bleeding events, particularly those that require blood transfusion, have a significant impact on the outcome and cost of treatment of patients with ACS. Transfusion rates in patients undergoing elective coronary artery bypass graft (CABG) surgery range from 30-60%, and transfusion in these patients is associated with increased short, medium and long-term mortality (Bracey et al., 1999; Engoren et al., 2002; Hebert et al., 1999). Bleeding is also the most frequent and costly complication associated with percutaneous coronary interventions (PCI), with transfusions being performed in 5-10% of patients at an incremental cost of $8000-$12,000 (Moscucci, 2002). In addition, the frequency of significant bleeding in patients undergoing treatment for ACS is high as well, ranging from 5% to 10% (excluding patients who undergo CABG), with bleeding and transfusion independently associated with a significant increase in short-term mortality (Moscucci et al., 2003; Rao et al., 2004). Therefore, despite the continued development of novel antithrombotics, a significant clinical need exists for safer anticoagulant agents.

Rapid reversal of drug activity can be achieved passively by formulation of a drug as an infusible agent with a short half-life with termination of infusion as the means to reverse, or actively via administration of a second agent, an antidote, that can neutralize the activity of the drug.

For hospitalized patients with acute ischemic heart disease, the ideal anticoagulant would be deliverable by intravenous or subcutaneous injection, immediately effective, easily dosed so as not to require frequent monitoring and immediately and predictably reversible.

### Current Approaches to Address the Problem

UFH is the only antidote-reversible anticoagulant currently approved for use. However, UFH has significant limitations. First, heparin has complex pharmacokinetics that make the predictability of its use challenging (Granger et al., 1996). Second, the dose predictability of its antidote, protamine, is challenging, and there are serious side effects associated with its use (Carr and Silverman, 1999; Welsby et al., 2005). Finally, heparin can induce thrombocytopenia (HIT) and thrombocytopenia with thrombosis (HITT) (Warkentin, 2005; Warkentin and Greinacher, 2004).

Despite these limitations, heparin remains the most commonly used anticoagulant for hospitalized patients primarily because it is “reversible.” Newer-generation anticoagulants, such as the LMWHs have improved upon the predictability of UFH dosing and do not require lab-based monitoring as part of their routine use. HIT and HITT are observed less frequently with the LMWHs, relative to UFH, but they have not eliminated this risk. Two of the three commercially available DTIs, lepirudin and argatroban, are specifically approved for use in patients who have developed or have a history of HIT. Bivalirudin is approved for use as an anticoagulant during PCI and therefore provides an attractive alternative to UFH in patients who have HIT. However, there are no direct and clear antidotes to reverse the anticoagulant effects of the LMWHs, nor of the DTIs, which presents a particular risk to their use in patients undergoing surgical or percutaneous coronary revascularization procedures (Jones et al., 2002). Bleeding in patients treated with LMWH's or DTI's is managed by administering blood products, including clotting factors.

### Blood Coagulation and FIX

The cell-based model of coagulation (FIG. 1) provides the clearest explanation to date of how physiologic coagulation occurs in vivo (Hoffman et al., 1995; Kjalke et al., 1998; Monroe et al., 1996).

According to this model, the procoagulant reaction occurs in three distinct steps, initiation, amplification and propagation. Initiation of coagulation takes place on tissue factor-bearing cells such as activated monocytes, macrophages, and endothelial cells. Coagulation factor VIIa, which forms a complex with tissue factor, catalyzes the activation of coagulation factors IX (FIX) and X (FX), which in turn generates a small amount of thrombin from prothrombin. In the amplification phase (also referred to as the priming phase), the small amount of thrombin generated in the initiation phase activates coagulation factors V, VIII, and XI and also activates platelets, which supplies a surface upon which further procoagulant reactions occur. In vivo, the small amounts of thrombin generated during the amplification phase are not sufficient to convert fibrinogen to fibrin, due to the presence of endogenous thrombin inhibitors termed serpins, such as anti-thrombin III, α-2-macroglobulin and heparin cofactor II. The final phase of the procoagulant reaction, propagation, occurs exclusively on the surface of activated platelets. During propagation, significant amounts of FIXa are generated by the FXIa-catalyzed activation of FIX. FIXa forms a complex with its requisite cofactor FVIIIa, which activates FX. Subsequently, FXa forms a complex with its requisite cofactor FVa. The FXa-FVa complex activates prothrombin, which leads to a “burst” of thrombin generation and fibrin deposition. The end result is the formation of a stable clot.

Based upon this model, FIXa play two roles in coagulation. In the initiation phase, FIXa plays an important role in generating small amounts of thrombin via activation of FX to FXa and subsequent prothrombin activation. However, this role of FIXa is at least partially redundant with the tissue factor FVIIa-catalyzed conversion of FX to FXa. The more critical role of FIXa occurs in the propagation phase, in which the FVIIIa/FIXa enzyme complex serves as the sole catalyst of FXa generation on the activated platelet surface. Therefore, a reduction in FIXa activity, either due to genetic deficiency in FIX (i.e. hemophilia B) or pharmacologic inhibition of FIX/IXa, is expected to have several effects on coagulation. First, inhibition or loss of FIXa activity should partially dampen the initiation of coagulation. Second, inhibition or loss of FIXa activity should have a profound effect on the propagation phase of coagulation, resulting in a significant reduction or elimination of thrombin production. Finally, limitation of thrombin generation during the propagation phase will at least partially quell feedback amplification of coagulation by reducing activation of platelets and upstream coagulation factors such as factors V, VIII and XI.

### Prior Animal and Human Evaluation of Inhibitors of FIXa

Inhibitors of FIX activity, such as active site-inactivated factor IXa (FIXai) or monoclonal antibodies against FIX (e.g., the antibody BC2), have exhibited potent anticoagulant and antithrombotic activity in multiple animal models, including various animal models of arterial thrombosis and stroke (Benedict et al., 1991; Choudhri et al., 1999; Feuerstein et al., 1999; Spanier et al., 1998a; Spanier et al., 1997; Spanier et al., 1998b; Toomey et al., 2000). In general, these studies have shown that FIXa inhibitors have a higher ratio of antithrombotic activity to bleeding risk than unfractionated heparin in animals. However, in these studies, at doses marginally higher than the effective dose, animals treated with these agents have exhibited bleeding profiles no different than heparin. Such an experience in well-controlled animal studies suggests that, in the clinical setting, the ability to control the activity of a FIXa inhibitor would enhance its safety and facilitate its medical use. In addition, FIXai has been shown to be safe and effective as a heparin replacement in multiple animal surgical models requiring anticoagulant therapy, including rabbit models of synthetic patch vascular repair, as well as canine and non-human primate models of CABG with cardiopulmonary bypass (Spanier et al., 1998a; Spanier et al., 1997; Spanier et al., 1998b). FIXai has also been used successfully for several critically ill patients requiring cardiopulmonary bypass and in the setting of other extracorporeal circuits such as extracorporeal membrane oxygenation (Spanier et al., 1998a) by physicians at the Columbia College of Physicians and Surgeons, on a compassionate care basis. Thus, FIxa is a validated target for anticoagulant therapy in coronary revascularization procedures (both CABG and PCI), and for the treatment and prevention of thrombosis in patients suffering from acute coronary syndromes.

Aptamer Drug Development, Drug-Antidote Pairs, and REG1

One approach to providing controlled anticoagulation is the utilization of an anticoagulation agent with medium- to long-term duration of action of ˜12 hours and greater that can achieve clinically appropriate activity at relatively low doses, in combination with a second agent capable of specifically binding to and neutralizing the primary anticoagulant. Such a “drug-antidote” combination can ensure predictable and safe neutralization and reversal of the anticoagulant activity of the drug (Rusconi et al., 2004, Nat Biotechnol. 22(11):1423-8; Rusconi et al., 2002, Nature 419(6902):90-4).

Applicants have applied the drug-antidote technology to the discovery of the REG1, aptamer based, anticoagulation system (see FIG. 2). Aptamers are single-stranded nucleic acids that bind with high affinity and specificity to target proteins (Nimjee et al., 2005), much like monoclonal antibodies. However, in order for an aptamer to bind to and inhibit a target protein, the aptamer must adopt a specific globular tertiary structure. Formation of this globular tertiary structure requires the aptamer to adopt the proper secondary structure (i.e., the correct base-paired and non-base-paired regions).

As shown in cartoon form in FIG. 2, introduction of an oligonucleotide complementary to a portion of an aptamer can change the aptamer's structure such that it can no longer bind to its target protein, and thus effectively reverses or neutralizes the pharmacologic activity of the aptamer drug (Rusconi et al., 2004, Nat Biotechnol. 22(11):1423-8; Rusconi et al., 2002, Nature 419(6902):90-4).

RB006 (P-L-guggaCUaUaCCgCgUaaUgCuGcCUccacT wherein P=mPEG2-NHS ester MW 40 kDa; L=C6 NH₂ linker; G=2-OH G; g=2′-O-Me G; C=2-F C; c=2′-O-Me C; U=2-F U; u=2′-O-Me U; a=2-O-Me A; and T=inverted 2′-H T (SEQ ID NO 1); see FIG. 2), the drug component of REG1, is a direct FIXa inhibitor that binds coagulation factor IXa with high affinity and specificity (Rusconi et al., 2004, Nat Biotechnol. 22(11):1423-8; Rusconi et al., 2002, Nature 419(6902):90-4; see also WO05/106042 to Duke University). RB006 elicits an anticoagulant effect by blocking the FVIIIa/FIXa-catalyzed conversion of FX to FXa. RB006 is a modified RNA aptamer, 31 nucleotides in length, which is moderately stabilized against endonuclease degradation by the presence of 2′-fluoro and 2′-O-methyl sugar-containing residues, and stabilized against exonuclease degradation by a 3′inverted deoxythymidine cap. The nucleic acid portion of the aptamer is conjugated to a 40-kilodalton polyethylene glycol (PEG) carrier to enhance its blood half-life. Following bolus IV injection, the half-life of RB006 in mice is approximately 8 hours and in monkeys, approximately 12 hours. As such, RB006 can be given as a one-time bolus injection, rather than by IV infusion, to maintain an anticoagulated state over several hours.

As shown in FIG. 2, RB007 (cgcgguauaguccac wherein g=2′-O-Me G; c=2′-O-Me C; u=2′-O-Me U; and a=2′-O-Me A (SEQ ID NO 2); see FIG. 2), the antidote component of REG1, is an oligonucleotide complementary to a portion of RB006 that can effectively bind to RB006 and thereby neutralize its anti-FIXa activity. RB007 is a 2′-O-methyl RNA oligonucleotide 15 nucleotides in length that is complementary to a portion of the drug component of REG1. The 2′-O-methyl modification confers moderate nuclease resistance to the antidote, which provides sufficient in vivo stability to enable it to seek and bind RB006, but does not support extended in vivo persistence.

### Nonclinical Development of REG1

Applicants have developed pharmacology data demonstrating the specificity of the RB006 aptamer for FIXa, and the affinity of the antidote RB007 for the aptamer. The results of the nonclinical pharmacology studies can be summarized as follows: the drug component of REG1 (RB006 and/or related precursor compounds) can: (1) effectively inhibit coagulation factor X activation in vitro; (2) prolong plasma clotting times in vitro in plasma from humans and other animal species; (3) systemically anticoagulate animals following bolus intravenous administration; (4) prevent thrombus formation in an animal arterial damage thrombosis model; (5) replace heparin in an animal cardiopulmonary bypass model, and (6) be effectively re-dosed in animals within 30 minutes following neutralization by the REG1 antidote component.

Nonclinical pharmacology studies to date have shown that the antidote component of REG1 (RB007 and/or antidotes specific to precursors of the REG1 drug component) can: (1) rapidly and durably neutralize the anticoagulant activity of the drug component of REG1 (RB006) in vitro in plasma from humans and other animal species; (2) rapidly and durably neutralize the anticoagulant activity of the drug component of REG1 in vivo following bolus IV administration in animals systemically anticoagulated with this agent; (3) prevent hemorrhage induced by a combination of supratherapeutic doses of the REG1 drug component and surgical trauma and (4) neutralize the anticoagulant activity of the REG1 drug component in animals following cardiopulmonary bypass. Furthermore, the antidote has not exhibited any anticoagulant or other pharmacologic activity in vitro in human plasma, or in animals following bolus IV administration.

There remains a need to provide a reliable method of administration which allows for the predictable and repeatable effect of an aptamer-antidote system.

### SUMMARY OF THE INVENTION

It has been found that there is a clear relationship between both the weight adjusted dose and, importantly, the body mass index-adjusted dose of an aptamer, in particular an aptamer anticoagulant, and its pharmacodynamic response. Furthermore, it was surprisingly found that the dose of an antidote to the aptamer need only be adjusted based on the amount of aptamer provided to the host, not on any additional criteria, to inhibit the activity of the aptamer to a desired level. This new understanding provides support for specific modes of administration that allow for predictable and repeatable dosing regimen for clinical use.

In one embodiment, the present invention provides an improved method of administration of an aptamer anticoagulant system comprising: 1) measuring the body mass index (BMI) of a host; 2) identifying a desired pharmacodynamic response; and 3) administering to the host a dose of an aptamer anticoagulant to achieve a desired pharmacodynamic response based on a comparison of the dose per BMI to pharmacodynamic response. In certain embodiments, an antidote to the aptamer is subsequently administered to the host where the dose of antidote is provided based on a ratio with the dose of aptamer previously administered adjusted for a desired reduction in aptamer activity. In certain instances, this dose of antidote is adjusted based on the time after administration of the aptamer. In certain instances, the ratio of antidote to aptamer is halved if the aptamer has been administered more than 24 hours previously.

In certain embodiments, a maximal level of anti-coagulation effect is desired. In these instances, an aptamer can be provided at a level of 4 mg/BMI or greater. In other instances, a level of anticoagulation of about 75% maximal is desired. In those instances, a dose of about between 0.75.0-1.5 mg/BMI is provided to the host. In other instances, a level of anticoagulation of about 50% maximal is desired. In these instances, a dose of about 0.25-0.5 mg/BMI is provided.

In certain general embodiments, the dosage of anticoagulant used is between 0.1 and 10 mg/BMI. In another embodiment, the dosage is between 0.2 and 8 mg/BMI, or between 0.2 and 6 mg/BMI, between 0.2 and 5 mg/BMI, between 0.2 and 4 mg/BMI, between 0.2 and 3 mg/BMI, between 0.2 and 2 mg/BMI, or between 0.2 and 1 mg/BMI. In some embodiments, the dose of anticoagulant is about 0.1 mg/BMI, or about 0.2 mg/BMI, or about 0.5 mg/BMI, or about 0.75 mg/BMI, or about 1 mg/BMI, or about 2 mg/BMI, or about 3 mg/BMI, or about 4 mg/BMI, or about 5 mg/BMI, or about 6 mg/BMI, or about 7 mg/BMI, or about 8 mg/BMI, or about 9 mg/BMI, or about 10 mg/BMI,

In another embodiment, the present invention provides an improved method of administration of an aptamer anticoagulant system comprising: 1) measuring the weight of a host; 2) identifying a desired pharmacodynamic response; and 3) administering to the host a dose of an aptamer anticoagulant to achieve a desired pharmacodynamic response based on a comparison of the dose per kilogram of host weight to pharmacodynamic response. In certain embodiments, an antidote to the aptamer is subsequently administered to the host where the dose of antidote is provided based on a ratio with the dose of aptamer previously administered adjusted for a desired reduction in aptamer activity. In certain instances, this dose of antidote is adjusted based on the time after administration of the aptamer. In certain instances, the ratio of antidote to aptamer is doubled if the aptamer has been administered more than 24 hours previously.

In certain embodiments, a maximal level of anti-coagulation effect is desired. In these instances, an aptamer can be provided at a level of 1.4 mg/kg or greater. In other instances, a level of anticoagulation of about 75% maximal is desired. In those instances, a dose of between 0.5 and 0.75 mg/kg is provided to the host. In other instances, a level of anticoagulation of about 50% maximal is desired. In these instances, a dose of about 0.2-0.4 mg/kg is provided.

In certain general embodiments, the dose used is between 0.1 and 2 mg/kg, between 0.1 and 1.8 mg/kg, between 0.1 and 1.6 mg/kg, between 0.1 and 1.5 mg/kg, between 0.1 and 1.4 mg/kg, between 0.1 and 1.3 mg/kg, between 0.1 and 1.2 mg/kg, between 0.1 and 1.1 mg/kg, between 0.1 and 1.0 mg/kg, between 0.1 and 0.9 mg/kg, between 0.1 and 0.8 mg/kg, between 0.1 and 0.7 mg/kg, between 0.1 and 0.6 mg/kg, between 0.1 and 0.5 mg/kg, between 0.1 and 0.4 mg/kg, between 0.1 and 0.3 mg/kg, or between 0.1 and 0.2 mg/kg. In other embodiments, the dose is between 1 and 20 mg/kg, between 1 and 18 mg/kg, between 1 and 15 mg/kg, between 2 and 15 mg/kg, between 3 and 15 mg/kg, between 4 and 15 mg/kg, between 5 and 20 mg/kg, between 5 and 15 mg/kg, or between 1 and 10 mg/kg, or between 5 and 10 mg/kg, or is about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, or about 10 mg/kg. In a principle embodiment, the aptamer anticoagulant system is the REG1 system, which comprises an aptamer anticoagulant and an oligonucleotide antidote. In certain, non-limiting embodiments, the aptamer is RB006 (SEQ ID NO 1) and the antidote is RB007 (SEQ ID NO 2). In one embodiment, the pharmacodynamic response is measured in coagulation assays such as the aPTT (plasma or whole blood) or the Activated Clotting Time (ACT), and can be reported as the absolute value, the percent effect, percent change, time weighted average or area under the curve over a defined time period.

The level of pharmacodynamic response can be at any level desired for a particular application. For example, in certain instances when a patient is at low risk for a thrombotic event, a low level of response may be desired. In particular instances, it may not be desirable to maximize clotting factor inhibition, and in particular FIX or FIXa inhibition by using a saturating amount of anticoagulant, particularly an aptamer to FIXa such as RB006. In other instances, when a patient is at a high risk for a thrombotic event or is having a thrombotic episode, a high level of response may be desired. In such instances, it may be desirable to maximize clotting factor inhibition, and in particular, FIX or FIXa inhibition by using a saturating amount of anticoagulant, particularly an aptamer to FIXa such as RB006.

In one embodiment, an anticoagulant aptamer, such as RB006, is provided in an IV bolus delivery. In another embodiment, an anticoagulant aptamer is provided by subcutaneous injection. In another embodiment, after IV or subcutaneous bolus delivery of the aptamer, an antidote is injected.

The procedures described herein allow for a step wise delivery of both anticoagulant and antidote to allow titration of either or both compounds to a desired level of target inhibition and reversal.

The ratio of antidote to aptamer is adjusted based on the desired level of inhibition of the aptamer. It was found that the antidote dose need only correlate to the dose of aptamer, and need not be additionally adjusted based on factors relating to the host. In one embodiment, the ratio of aptamer to antidote is 1:1. In other embodiments, the ratio of aptamer to antidote is greater than 1:1 such as 2:1, 3:1, 4:1, 5:1, 6:1, 7:1, 8:1, 9:1, 10:1 or more. These ratios can also be calculated based on antidote to aptamer ratio, which can, for example, be less than about 1:1 such as 0.9:1 or about 0.9:1, 0.8:1 or about 0.8:1, 0.7:1 or about 0.7:1, 0.6:1 or about 0.6:1, 0.5:1 or about 0.5:1, 0.45:1 or about 0.45:1, 0.4:1 or about 0.4:1, 0.35:1 or about 0.35:1, 0.3:1 or about 0.3:1, 0.25:1 or about 0.25:1, 0.2:1 or about 0.2:1, 0.15:1 or about 0.15:1, 0.1:1 or about 0.1:1 or less than 0.1:1 such as about 0.005:1 or less. In some embodiments, the ratio is between 0.5:1 and 0.1:1, or between 0.5:1 and 0.2:1, or between 0.5:1 and 0.3:1. In other embodiments, the ratio is between 1:1 and 5:1, or between 1:1 and 10:1, or between 1:1 and 20:1.

In some embodiments, only a partial reversal of aptamer activity occurs. For example, in some embodiments, aptamer activity is reversed by 90%, or less than 90% such as about 80%, about 70%, about 60%, about 50%, about 40%, about 30%, about 20%, about 10% or less. The ratio of antidote to aptamer can be calculated either by comparing weight to weight or on a molar basis.

In particular embodiments of the invention, the host or subject to which the dosing system is applied is a human. In specific embodiments, the host is a human who is in need of anticoagulant therapy. In certain embodiments, the host is a human patient undergoing vascular surgery, such as CABG surgery.

### BRIEF DESCRIPTION OF THE FIGURES

FIG. 1 depicts cell based model of coagulation. TF—tissue factor; vWF—von Willebrands factor; II—prothrombin; IIa—thrombin; Va, VIIa, VIIIa, IXa, Xa, XIa—activated forms of coagulation factors V, VII, VIII, IX, X and XI.

FIG. 2 depicts the REG1 anticoagulation system. The system is composed of the FIXa inhibitor RB006 and its matched antidote RB007. Recognition of the drug by the antidote is via Watson-Crick base pairing as shown. RB006 is a modified RNA aptamer composed of 2′-fluoro residues (upper case) 2′-O-methyl residues (lower case) and a single 2′-hydroxyl residue (underlined). RB006 is conjugated to a 40-KDa polyethylene glycol carrier (P) via a 6-carbon amino linker (L), and is protected from exonuclease degradation by an inverted deoxythymidine on the 3′ end (idT). RB007 (the antidote) is a 2′-O-methyl-modified RNA oligonucleotide.

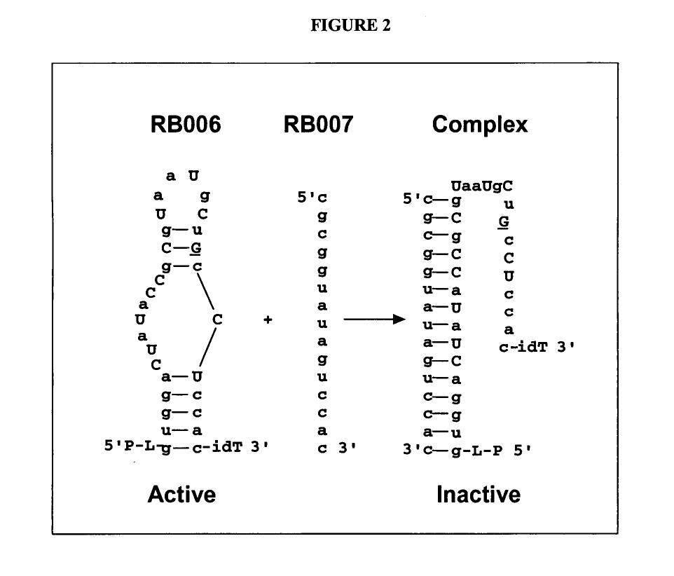

FIG. 3 is a graph of RB006 APTT dose response curve in vitro showing that RB006 elicits a concentration-dependent increase in the APTT of normal pooled human plasma. “Mean Sec” is the mean APTT. Data were fit to a four parameter logistic equation, allowing for determination of the IC50 of the curve.

FIG. 4 is a graph of RB006 anticoagulant effect in plasma from individuals. The anticoagulant activity of RB006 was measured in 4 individuals, two females and two males. Plasma samples were obtained from George King Biomedical (Overland Park, Kans.). Individuals were screened and confirmed normal with respect to coagulation factor levels. M/55 connotes the donor was a male, age 55 years; F/49 connotes the donor was a female, age 49 years. APTT reagent used is MDA Platelin L (Biomeriux), which is relatively more sensitive to FIX levels than the APTT reagent used in the study presented in FIG. 3.

FIG. 5 is a graph showing drug neutralization activity of antidote RB007. A low molar excess of antidote RB007 to aptamer RB006 completely neutralizes the anticoagulant activity of RB006 within 10 minutes. Data shown are the mean±SEM from three independent measurements. The molar ratio is based on the moles of oligonucleotide for the aptamer and antidote (AD).

FIG. 6 is a graph of re-dosing of aptamer RB006 following antidote neutralization of prior drug dose. Pigs were administered 2.5 mg/kg aptamer RB006 and, 15 minutes later, were treated with 3 mg/kg RB007 antidote (n=2) to neutralize this initial dose. Then, 30 minutes after antidote RB007 administration (45 minutes post initial aptamer dosing), pigs were re-dosed with 2.5 mg/kg aptamer RB006. The change in clot time was measured in (A) ACT (◯) assays in whole blood; or (B) APTT (◯) clotting assays in plasma. Data shown are the mean±the range for duplicate measurements from each animal. The bold line in (A and B) is a simple point-to-point line through the data points.

FIG. 7 is a graph of RB006 in vitro APTT Dose Response Curve in Plasma from Cynomolgus Monkeys and Humans. RB006 elicits a dose-dependent prolongation of APTT in plasma from monkeys that is very similar to that observed in human plasma. Experiments were performed using the same brand of APTT reagent, APTT-LS, as used to analyze plasma samples in the nonclinical toxicity studies performed in monkeys (REG1-TOX001 and REG1-TOX003). Therefore, these data serve as a basis for interpreting the APTT results from REG1-TOX001 and REG1-TOX003 presented in Sections 8.4. According to the manufacturer (Pacific Hemostasis, Middletown, Va.), this reagent yields an APTT of ˜87.3 seconds in human plasma samples containing <1% FIX levels, 36.1 seconds in samples containing ˜20% normal FIX activity, and 27.5 seconds in samples containing 100% FIX activity. Citrated, pooled cynomolgus monkey plasma was provided by Charles River Laboratories, Sierra Division.

FIG. 8 is a graph of systemic anticoagulation of monkeys by RB006 administration. The level of anticoagulation in the monkeys was monitored with the APTT. For animals treated with 15 mg/kg, RB 006 data are presented as the mean±SEM. For animals at the 5 and 30-mg/kg dose levels, data are presented as the mean±range, as there were only 2 animals at each of these dose levels.

FIG. 9 is a graph of systemic anticoagulation of monkeys with RB006 and reversal with antidote RB007. The level of anticoagulation in the monkeys was monitored with the APTT. RB007 was administered at t=3 hours following RB006 administration. Data are presented as the mean±SEM.

FIG. 10 is a graph of pharmacodynamic activity of RB006 in Humans

FIG. 11 is a graph of the neutralization of the pharmacologic activity of RB006 in humans by RB007

FIG. 11 is a graph comparing the pharmacodynamic activity of RB 006 with and without RB007 administration

FIG. 12 is a graph comparing the pharmacodynamic response in subjects treated with 60 mg RB006 followed by treatment with RB007 versus placebo at 3 hours

FIG. 13 shows a more detailed analysis of the relative increase in APTT over baseline from 0-3 hrs for all subjects who received RB006.

FIG. 14 is a graph of the AUC 0-3 for each subject organized by RB006 dose level (15, 30, 60 or 90 mg). Because the relative effect is being measured over 3 hrs, a value of “3” represents no response to RB006, a value of 6 indicates an average 2 fold increase over baseline, etc.

FIG. 15 is a graph of the weight-adjusted dose of RB006 as a function of RB006 dose level.

FIG. 16 is a graph of the AUC0-3 compared to the “weight adjusted” dose of RB006.

FIG. 17 is a graph of the BMI adjusted dose of subjects treated with RB006 as a function of RB006 dose level.

FIG. 18 is a graph AUC0-3 for RB006 versus BMI adjusted dose.

FIG. 19 is a graph of APTT compared to baseline relative to % FIX activity showing the APTT at different doses of RB006 (15, 30, 60 and 90 mg).

FIG. 20 is a graph of APTT response compared using four doses of RB006 aptamer and RB007 antidote administered IV in patients with coronary artery disease.

FIG. 21 is a graph showing the time weighted APTT after RB006 (0.75 mg/kg) administration at days 1, 3 and 5 in all treatment groups. Group 1: subjects received a single dose of the aptamer (0.75 mg/kg RB006) on Days 1, 3, and 5, followed by a fixed-dose of antidote (1.5 mg/kg RB007) one hour later; Groups 2 and 3: subjects received a single dose of aptamer RB006 (0.75 mg/kg) on Days 1, 3, and 5, followed by varying single doses of RB007 administered one hour later.

FIG. 23 is a graph of mean APTT over time in groups administered RB006 (0.75 mg/kg) and RB007 at various ratios compared to RB006.

FIG. 24 is a graph showing the percent recover in teim weighted APTT from administration of RB006 after administration, at one hour, of RB007 at listed ratios when compared to RB006.

### DETAILED DESCRIPTION

It has been found that there is a clear relationship between both the weight adjusted dose and, importantly, the body mass index-adjusted dose of an aptamer, in particular an aptamer anticoagulant, and its pharmacodynamic response. Furthermore, it was surprisingly found that the dose of an antidote to the aptamer need only be adjusted based on the amount of aptamer provided to the host, not on any additional criteria, to inhibit the activity of the aptamer to a desired level. This new understanding provides support for specific modes of administration that allow for predictable and repeatable dosing regimen for clinical use.

### Development of Aptamers

Nucleic acid aptamers are isolated using the Systematic Evolution of Ligands by EXponential Enrichment, termed SELEX, process. This method allows the in vitro evolution of nucleic acid molecules with highly specific binding to target molecules. The SELEX method is described in, for example, U.S. Pat. No. 7,087,735, U.S. Pat. No. 5,475,096 and U.S. Pat. No. 5,270,163, (see also WO 91/19813).

The SELEX method involves selection from a mixture of candidate oligonucleotides and step-wise iterations of binding, partitioning and amplification, using the same general selection scheme, to achieve virtually any desired criterion of binding affinity and selectivity. Starting from a mixture of nucleic acids, such as mixtures comprising a segment of randomized sequence, the SELEX method includes steps of contacting the mixture with the target under conditions favorable for binding, partitioning unbound nucleic acids from those nucleic acids which have bound specifically to target molecules, dissociating the nucleic acid-target complexes, amplifying the nucleic acids dissociated from the nucleic acid-target complexes to yield a ligand-enriched mixture of nucleic acids, then reiterating the steps of binding, partitioning, dissociating and amplifying through as many cycles as desired to yield highly specific, high affinity aptamers to the target molecule.

The basic SELEX method has been modified to achieve a number of specific objectives. For example, U.S. Pat. No. 5,707,796 describes the use of SELEX in conjunction with gel electrophoresis to select nucleic acid molecules with specific structural characteristics, such as bent DNA. U.S. Pat. No. 5,763,177 describes a SELEX-based method for selecting aptamers containing photoreactive groups capable of binding and/or photocrosslinking to and/or photoinactivating a target molecule. U.S. Pat. No. 5,580,737 describes a method for identifying highly specific aptamers able to discriminate between closely related molecules, termed Counter-SELEX. U.S. Pat. Nos. 5,567,588 and 5,861,254 describe SELEX-based methods which achieve highly efficient partitioning between oligonucleotides having high and low affinity for a target molecule. U.S. Pat. No. 5,496,938, describes methods for obtaining improved aptamers after the SELEX process has been performed. U.S. Pat. No. 5,705,337, describes methods for covalently linking a ligand to its target.

The feasibility of identifying aptamers to small peptides in solution was demonstrated in U.S. Pat. No. 5,648,214. The ability to use affinity elution with a ligand to produce aptamers that are targeted to a specific site on the target molecule is exemplified in U.S. Pat. No. 5,780,228, which relates to the production of high affinity aptamers binding to certain lectins. Methods of preparing aptamers to certain tissues, which include groups of cell types, are described in U.S. Pat. No. 6,127,119. The production of certain modified high affinity ligands to calf intestinal phosphatase is described in U.S. Pat. No. 6,673,553. U.S. Pat. No. 6,716,580 describes an automated process of identifying aptamers that includes the use of a robotic manipulators.

In its most basic form, the SELEX process may be defined by the following series of steps:

1) A candidate mixture of nucleic acids of differing sequence is prepared. The candidate mixture generally includes regions of fixed sequences (i.e., each of the members of the candidate mixture contains the same sequences in the same location) and regions of randomized sequences. The fixed sequence regions are selected either: (a) to assist in the amplification steps described below, (b) to mimic a sequence known to bind to the target, or (c) to enhance the concentration of a given structural arrangement of the nucleic acids in the candidate mixture. The randomized sequences can be totally randomized (i.e., the probability of finding a base at any position being one in four) or only partially randomized (e.g., the probability of finding a base at any location can be selected at any level between 0 and 100 percent).

2) The candidate mixture is contacted with the selected target under conditions favorable for binding between the target and members of the candidate mixture. Under these circumstances, the interaction between the target and the nucleic acids of the candidate mixture can be considered as forming nucleic acid-target pairs between the target and those nucleic acids having the strongest affinity for the target.

3) The nucleic acids with the highest affinity for the target are partitioned from those nucleic acids with lesser affinity to the target. Because only an extremely small number of sequences (and possibly only one molecule of nucleic acid) corresponding to the highest affinity nucleic acids exist in the candidate mixture, it is generally desirable to set the partitioning criteria so that a significant amount of the nucleic acids in the candidate mixture (approximately 5 to 50%) are retained during partitioning.

4) Those nucleic acids selected during partitioning as having the relatively higher affinity to the target are then amplified to create a new candidate mixture that is enriched in nucleic acids having a relatively higher affinity for the target.

5) By repeating the partitioning and amplifying steps above, the newly formed candidate mixture contains fewer and fewer weakly binding sequences, and the average degree of affinity of the nucleic acids to the target will generally increase. Taken to its extreme, the SELEX process will yield a candidate mixture containing one or a small number of unique nucleic acids representing those nucleic acids from the original candidate mixture having the highest affinity to the target molecule.

### Chemical Modifications

One problem encountered in the therapeutic use of nucleic acids is that oligonucleotides in their phosphodiester form may be quickly degraded in body fluids by intracellular and extracellular enzymes such as endonucleases and exonucleases before the desired effect is manifest. Certain chemical modifications of the aptamer can be made to increase the in vivo stability of the aptamer or to enhance or to mediate the delivery of the aptamer.

Modifications of the aptamers include, but are not limited to, those which provide other chemical groups that incorporate additional charge, polarizability, hydrophobicity, hydrogen bonding, electrostatic interaction, and fluxionality to the aptamer bases or to the aptamer as a whole. Such modifications include, but are not limited to, 2′-position sugar modifications, 5-position pyrimidine modifications, 8-position purine modifications, modifications at exocyclic amines, substitution of 4-thiouridine, substitution of 5-bromo or 5-iodo-uracil; backbone modifications, phosphorothioate or alkyl phosphate modifications, methylations, unusual base-pairing combinations such as the isobases isocytidine and isoguanidine and the like. Modifications can also include 3′ and 5′ modifications such as capping.

The SELEX method encompasses the identification of high-affinity aptamers containing modified nucleotides conferring improved characteristics on the ligand, such as improved in vivo stability or improved delivery characteristics. Examples of such modifications include chemical substitutions at the ribose and/or phosphate and/or base positions. SELEX-identified aptamers containing modified nucleotides are described in U.S. Pat. No. 5,660,985 that describes oligonucleotides containing nucleotide derivatives chemically modified at the 5- and 2′-positions of pyrimidines. U.S. Pat. No. 5,580,737 describes specific aptamers containing one or more nucleotides modified with 2′-amino (2′-NH2), 2′-fluoro (2′-F), and/or 2′-O-methyl (2′-OMe). U.S. Pat. No. 5,756,703, describes oligonucleotides containing various 2′-modified pyrimidines.

The SELEX method encompasses combining selected oligonucleotides with other selected oligonucleotides and non-oligonucleotide functional units as described in U.S. Pat. Nos. 5,637,459 and 5,683,867. U.S. Pat. No. 5,637,459 describes highly specific aptamers containing one or more nucleotides modified with 2′-amino (2′-NH 2), 2′-fluoro (2′-F), and/or 2′-O-methyl (2′-OMe). The SELEX method further encompasses combining selected aptamers with lipophilic or Non-Immunogenic, High Molecular Weight compounds in a diagnostic or therapeutic complex as described in U.S. Pat. No. 6,011,020.

Where the aptamers are derived by the SELEX method, the modifications can be pre- or post-SELEX modifications. Pre-SELEX modifications can yield aptamers with both specificity for its target and improved in vivo stability. Post-SELEX modifications made to 2′-OH aptamers can result in improved in vivo stability without adversely affecting the binding capacity of the aptamers. In one embodiment, the modifications of the aptamer include a 3′-3′ inverted phosphodiester linkage at the 3′ end of the molecule and 2′ fluoro (2′-F) and/or 2′ amino (2′-NH2), and/or 2′ O methyl (2′-OMe) modification of some or all of the nucleotides.

In one embodiment, the aptamer or its regulator can be covalently attached to a lipophilic compound such as cholesterol, dialkyl glycerol, diacyl glycerol, or a non-immunogenic, high molecular weight compound or polymer such as polyethylene glycol (PEG). In these cases, the pharmacokinetic properties of the aptamer or modulator can be enhanced. In still other embodiments, the aptamer or the modulator can be encapsulated inside a liposome. The lipophilic compound or non-immunogenic, high molecular weight compound can be covalently bonded or associated through non-covalent interactions with aptamer or modulator(s). In embodiments where covalent attachment is employed, the lipophilic compound or non-immunogenic, high molecular weight compound may be covalently bound to a variety of positions on the aptamer or modulator, such as to an exocyclic amino group on the base, the 5-position of a pyrimidine nucleotide, the 8-position of a purine nucleotide, the hydroxyl group of the phosphate, or a hydroxyl group or other group at the 5′ or 3′ terminus. In one embodiment, the covalent attachment is to the 5′ or 3′ hydroxyl group. Attachment of the oligonucleotide modulator to other components of the complex can be done directly or with the utilization of linkers or spacers.

Oligonucleotides of the invention can be modified at the base moiety, sugar moiety, or phosphate backbone, for example, to improve stability of the molecule, hybridization, etc. The oligonucleotide can include other appended groups. To this end, the oligonucleotide can be conjugated to another molecule, e.g., a peptide, hybridization triggered cross-linking agent, transport agent, hybridization-triggered cleavage agent, etc. The oligonucleotide can comprise at least one modified base moiety which is selected from the group including, but not limited to, 5-fluorouracil, 5-bromouracil, 5-chlorouracil, 5-iodouracil, hypoxanthine, xanthine, 4-acetylcytosine, 5-(carboxyhydroxymethyl) uracil, 5-carboxymethylaminomethyl thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2α-thiouracil, β-D-mannosylqueosine, 5′-methoxycarboxymethyluracil, 5-methoxyuracil, 2-methylthio-N&isopentenyladenine, uracil oxyacetic acid, wybutoxosine, pseudouracil, queosine, 2-thiocytosine, 5-methyl thiouracil, 2-thiouracil, 4-thiouracil, 5-methyluracil, -uracil-5-oxyacetic acid methylester, uracil oxyacetic acid (v), 5-methyl thiouracil, 3-(3-amino-3-N carboxypropyl) and 2,6-diaminopurine.

An aptamer or modulator of the invention can also comprise at least one modified sugar moiety selected from the group including, but not limited to, arabinose, 2-fluoroarabinose, xylose, and hexose. The aptamer or modulator can comprise at least one modified phosphate backbone selected from the group including, but not limited to, a phosphorothioate, a phosphorodithioate, a phosphoramidothioate, a phosphoramidate, a phosphorodiamidate, a methylphosphonate, an alkyl phosphotriester, and a formacetal or analog thereof.

Any of the oligonucleotides of the invention can be synthesized by standard methods known in the art, e.g. by use of an automated DNA synthesizer (such as are commercially available from, for example, Biosearch, Applied Biosystems).

### Modulators

The modulators of the invention can be oligonucleotides, small molecules, peptides, oligosaccharides, for example aminoglycosides, or other molecules that can bind to or otherwise modulate the activity of the aptamer, or a chimera or fusion or linked product of any of these.

In one embodiment, the modulator is an oligonucleotide complementary to at least a portion of the aptamer. In another embodiment, the modulator can be a ribozyme or DNAzyme that targets the aptamer. In a further embodiment, the modulator can be a peptide nucleic acid (PNA), morpholino nucleic acid (MNA), locked nucleic acid (LNA) or pseudocyclic oligonucleobases (PCO) that includes a sequence that is complementary to or hybridizes with at least a portion of the aptamer.

An aptamer possesses an active tertiary structure which is dependent on formation of the appropriate stable secondary structure. Therefore, while the mechanism of formation of a duplex between a complementary oligonucleotide modulator of the invention and an aptamer is the same as between two short linear oligoribonucleotides, both the rules for designing such interactions and the kinetics of formation of such a product are impacted by the intramolecular aptamer structure. The rate of nucleation is important for formation of the final stable duplex, and the rate of this step is greatly enhanced by targeting the oligonucleotide modulator to single-stranded loops and/or single-stranded 3′ or 5′ tails present in the aptamer. For the formation of the intermolecular duplex to occur, the free energy of formation of the intermolecular duplex has to be favorable with respect to formation of the existing intramolecular duplexes within the targeted aptamer.

Modulators can be designed so as to bind a particular aptamer with a high degree of specificity and a desired degree of affinity. Modulators can be also be designed so that, upon binding, the structure of the aptamer is modified to either a more or less active form. For example, the modulator can be designed so that upon binding to the targeted aptamer, the three-dimensional structure of that aptamer is altered such that the aptamer can no longer bind to its target molecule or binds to its target molecule with less affinity.

Alternatively, the modulator can be designed so that, upon binding, the three dimensional structure of the aptamer is altered so that the affinity of the aptamer for its target molecule is enhanced. That is, the modulator can be designed so that, upon binding, a structural motif is produced in the aptamer so that the aptamer can bind to its target molecule.

In an alternative embodiment of the invention, the modulator itself is an aptamer. In this embodiment, a aptamer is first generated that binds to the desired therapeutic target. In a second step, a second aptamer that binds to the first aptamer is generated using the SELEX process described herein or other process, and modulates the interaction between the therapeutic aptamer and the target. In one embodiment, the second aptamer deactivates the effect of the first aptamer.

In other alternative embodiments, the aptamer which binds to the target can be a PNA, MNA, LNA or PCO and the modulator is a aptamer. Alternatively, the aptamer which binds to the target is a PNA, MNA, LNA or PCO, and the modulator is a PNA. Alternatively, the aptamer which binds to the target is a PNA, MNA, LNA or PCO, and the modulator is an MNA. Alternatively, the aptamer which binds to the target is a PNA, MNA, LNA or PCO, and the modulator is an LNA. Alternatively, the aptamer which binds to the target is a PNA, MNA, LNA or PCO, and the modulator is a PCO. Any of these can be used, as desired, in the naturally occurring stereochemistry or in non-naturally occurring stereochemistry or a mixture thereof. For example, in a preferred embodiment, the aptamer is in the D configuration, and in an alternative embodiment, the aptamer is in the L configuration.

In one embodiment, the modulator of the invention is an oligonucleotide that comprises a sequence complementary to at least a portion of the targeted aptamer sequence. For example, the modulator oligonucleotide can comprise a sequence complementary to 6-25 nucleotides of the targeted aptamer, typically, 8-20 nucleotides, more typically, 10-15 nucleotides. Advantageously, the modulator oligonucleotide is complementary to 6-25 consecutive nucleotides of the aptamer, or 8-20 or 10-15 consecutive nucleotides. The length of the modulator oligonucleotide can be optimized taking into account the targeted aptamer and the effect sought. Typically the modulator oligonucleotide is 5-80 nucleotides in length, more typically, 10-30 and most typically 15-20 nucleotides (e.g., 15-17). The oligonucleotide can be made with nucleotides bearing D or L stereochemistry, or a mixture thereof. Naturally occurring nucleosides are in the D configuration.

Various strategies can be used to determine the optimal site for oligonucleotide binding to a targeted aptamer. An empirical strategy can be used in which complimentary oligonucleotides are “walked” around the aptamer. A walking experiment can involve two experiments performed sequentially. A new candidate mixture can be produced in which each of the members of the candidate mixture has a fixed nucleic acid-region that corresponds to a oligonucleotide modulator of interest. Each member of the candidate mixture also contains a randomized region of sequences. According to this method it is possible to identify what are referred to as “extended” aptamers, which contain regions that can bind to more than one binding domain of an aptamer. In accordance with this approach, 2′-O-methyl oligonucleotides (e.g., 2′-O-methyl oligonucleotides) about 15 nucleotides in length can be used that are staggered by about 5 nucleotides on the aptamer (e.g., oligonucleotides complementary to nucleotides 1-15, 6-20, 11-25, etc. of aptamer the aptamer). An empirical strategy can be particularly effective because the impact of the tertiary structure of the aptamer on the efficiency of hybridization can be difficult to predict. Assays described in the Examples that follow can be used to assess the ability of the different oligonucleotides to hybridize to a specific aptamer, with particular emphasis on the molar excess of the oligonucleotide required to achieve complete binding of the aptamer. The ability of the different oligonucleotide modulators to increase the rate of dissociation of the aptamer from, or association of the aptamer with, its target molecule can also be determined by conducting standard kinetic studies using, for example, BIACORE assays. Oligonucleotide modulators can be selected such that a 5-50 fold molar excess of oligonucleotide, or less, is required to modify the interaction between the aptamer and its target molecule in the desired manner.

Alternatively, the targeted aptamer can be modified so as to include a single-stranded tail (3′ or 5′) in order to promote association with an oligonucleotide modulator. Suitable tails can comprise 1 to 20 nucleotides, preferably, 1-10 nucleotides, more preferably, 1-5 nucleotides and, most preferably, 3-5 nucleotides (e.g., modified nucleotides such as 2′-O-methyl sequences). Tailed aptamers can be tested in binding and bioassays (e.g., as described in the Examples that follow) to verify that addition of the single-stranded tail does not disrupt the active structure of the aptamer. A series of oligonucleotides (for example, 2′-O-methyl oligonucleotides) that can form, for example, 1, 3 or 5 base pairs with the tail sequence can be designed and tested for their ability to associate with the tailed aptamer alone, as well as their ability to increase the rate of dissociation of the aptamer from, or association of the aptamer with, its target molecule. Scrambled sequence controls can be employed to verify that the effects are due to duplex formation and not non-specific effects.

The oligonucleotide modulators of the invention comprise a sequence complementary to at least a portion of a aptamer. However, absolute complementarity is not required. A sequence “complementary to at least a portion of an aptamer,” referred to herein, means a sequence having sufficient complementarity to be able to hybridize with the aptamer. The ability to hybridize can depend on both the degree of complementarity and the length of the antisense nucleic acid. Generally, the larger the hybridizing oligonucleotide, the more base mismatches with a target aptamer it can contain and still form a stable duplex (or triplex as the case may, be). One skilled in the art can ascertain a tolerable degree of mismatch by use of standard procedures to determine the melting point of the hybridized complex. In specific aspects, the oligonucleotide can be at least 5 or at least 10 nucleotides, at least 15 or 17 nucleotides, at least 25 nucleotides or at least 50 nucleotides. The oligonucleotides of the invention can be DNA or RNA or chimeric mixtures or derivatives or modified versions thereof, single-stranded.

In one embodiment, the modulator is a ribozyme or a DNAzyme. There are at least five classes of ribozymes that each display a different type of specificity. For example, Group I Introns are about 300 to >1000 nucleotides in size and require a U in the target sequence immediately 5′ of the cleavage site and binds 4-6 nucleotides at the 5′-side of the cleavage site. Another class are RNaseP RNA (M1 RNA), which are about 290 to 400 nucleotides in size. A third example are Hammerhead Ribozyme, which are about 30 to 40 nucleotides in size. They require the target sequence UH immediately 5′ of the cleavage site and bind a variable number nucleotides on both sides of the cleavage site. A fourth class are the Hairpin Ribozymes, which are about 50 nucleotides in size. They requires the target sequence GUC immediately 3′ of the cleavage site and bind 4 nucleotides at the 5′-side of the cleavage site and a variable number to the 3′-side of the cleavage site. The fifth group are Hepatitis Delta Virus (HDV) Ribozymes, which are about 60 nucleotides in size.

Another class of catalytic molecules are called “DNAzymes”. DNAzymes are single-stranded, and cleave both RNA and DNA. A general model for the DNAzyme has been proposed, and is known as the “10-23” model. DNAzymes following the “10-23” model have a catalytic domain of 15 deoxyribonucleotides, flanked by two substrate-recognition domains of seven to nine deoxyribonucleotides each.

Nucleobases of the oligonucleotide modulators of the invention can be connected via internucleobase linkages, e.g., peptidyl linkages (as in the case of peptide nucleic acids (PNAs); Nielsen et al. (1991) Science 254, 1497 and U.S. Pat. No. 5,539,082) and morpholino linkages (Qin et al., Antisense Nucleic Acid Drug Dev. 10, 11 (2000); Summerton, Antisense Nucleic Acid Drug Dev. 7, 187 (1997); Summerton et al., Antisense Nucleic Acid Drug Dev. 7, 63 (1997); Taylor et al., J Biol Chem. 271, 17445 (1996); Partridge et al., Antisense Nucleic Acid Drug Dev. 6, 169 (1996)), or by any other natural or modified linkage. The oligonucleobases can also be Locked Nucleic Acids (LNAs). Nielsen et al., J Biomol Struct Dyn 17, 175 (1999); Petersen et al., J Mol Recognit 13, 44 (2000); Nielsen et al., Bioconjug Chem 11, 228 (2000).

PNAs are compounds that are analogous to oligonucleotides, but differ in composition. In PNAs, the deoxyribose backbone of oligonucleotide is replaced with a peptide backbone. Each subunit of the peptide backbone is attached to a naturally-occurring or non-naturally-occurring nucleobase. PNA often has an achiral polyamide backbone consisting of N-(2-aminoethyl)glycine units. The purine or pyrimidine bases are linked to each unit via a methylene carbonyl linker (1-3) to target the complementary nucleic acid. PNA binds to complementary RNA or DNA in a parallel or antiparallel orientation following the Watson-Crick base-pairing rules. The uncharged nature of the PNA oligomers enhances the stability of the hybrid PNA/DNA(RNA) duplexes as compared to the natural homoduplexes.

Morpholino nucleic acids are so named because they are assembled from morpholino subunits, each of which contains one of the four genetic bases (adenine, cytosine, guanine, and thymine) linked to a 6-membered morpholine ring. Eighteen to twenty-five subunits of these four subunit types are joined in a specific order by non-ionic phosphorodiamidate intersubunit linkages to give a morpholino oligo. These morpholino oligos, with their 6-membered morpholine backbone moieties joined by non-ionic linkages, afford substantially better antisense properties than do RNA, DNA, and their analogs having 5-membered ribose or deoxyribose backbone moieties joined by ionic linkages (see wwwgene-tools.com/Morphol-inos/body_morpholinos.HTML).

LNA is a class of DNA analogues that possess some features that make it a prime candidate for modulators of the invention. The LNA monomers are bi-cyclic compounds structurally similar to RNA-monomers. LNA share most of the chemical properties of DNA and RNA, it is water-soluble, can be separated by gel electrophoreses, ethanol precipitated etc (Tetrahedron, 54, 3607-3630 (1998)). However, introduction of LNA monomers into either DNA or RNA oligos results in high thermal stability of duplexes with complementary DNA or RNA, while, at the same time obeying the Watson-Crick base-pairing rules. This high thermal stability of the duplexes formed with LNA oligomers together with the finding that primers containing 3′ located LNA(s) are substrates for enzymatic extensions, e.g. the PCR reaction, is used in the present invention to significantly increase the specificity of detection of variant nucleic acids in the in vitro assays described in the application. The amplification processes of individual alleles occur highly discriminative (cross reactions are not visible) and several reactions may take place in the same vessel. See for example U.S. Pat. No. 6,316,198.

Pseudo-cyclic oligonucleobases (PCOs) can also be used as a modulator in the present invention (see U.S. Pat. No. 6,383,752). PCOs contain two oligonucleotide segments attached through their 3′-3′ or 5′-5′ ends. One of the segments (the “functional segment”) of the PCO has some functionality (e.g., an antisense oligonucleotide complementary to a target mRNA). Another segment (the “protective segment”) is complementary to the 3′- or 5′-terminal end of the functional segment (depending on the end through which it is attached to the functional segment). As a result of complementarity between the functional and protective segment segments, PCOs form intramolecular pseudo-cyclic structures in the absence of the target nucleic acids (e.g., RNA). PCOs are more stable than conventional antisense oligonucleotides because of the presence of 3′-3′ or 5′-5′ linkages and the formation of intramolecular pseudo-cyclic structures. Pharmacokinetic, tissue distribution, and stability studies in mice suggest that PCOs have higher in vivo stability than and, pharmacokinetic and tissue distribution profiles similar to, those of PS-oligonucleotides in general, but rapid elimination from selected tissues. When a fluorophore and quencher molecules are appropriately linked to the PCOs of the present invention, the molecule will fluoresce when it is in the linear configuration, but the fluorescence is quenched in the cyclic conformation.

Peptide-based modulators of aptamers represent an alternative molecular class of modulators to oligonucleotides or their analogues. This class of modulators are particularly prove useful when sufficiently active oligonucleotide modulators of a target aptamer can not be isolated due to the lack of sufficient single-stranded regions to promote nucleation between the target and the oligonucleotide modulator. In addition, peptide modulators provide different bioavailabilities and pharmacokinetics than oligonucleotide modulators.

Oligosaccharides, like aminoglycosides, can bind to nucleic acids and can be used to modulate the activity of aptamers. A small molecule that intercalates between the aptamer and the target or otherwise disrupts or modifies the binding between the aptamer and target can also be used as the therapeutic regulator. Such small molecules can be identified by screening candidates in an assay that measures binding changes between the aptamer and the target with and without the small molecule, or by using an in vivo or in vitro assay that measures the difference in biological effect of the aptamer for the target with and without the small molecule. Once a small molecule is identified that exhibits the desired effect, techniques such as combinatorial approaches can be used to optimize the chemical structure for the desired regulatory effect.

Standard binding assays can be used to identify and select modulators of the invention. Nonlimiting examples are gel shift assays and BIACORE assays. That is, test modulators can be contacted with the aptamers to be targeted under test conditions or typical physiological conditions and a determination made as to whether the test modulator in fact binds the aptamer. Test modulators that are found to bind the aptamer can then be analyzed in an appropriate bioassay (which will vary depending on the aptamer and its target molecule, for example coagulation tests) to determine if the test modulator can affect the biological effect caused by the aptamer on its target molecule.

The Gel-Shift assay is a technique used to assess binding capability. For example, a DNA fragment containing the test sequence is first incubated with the test protein or a mixture containing putative binding proteins, and then separated on a gel by electrophoresis. If the DNA fragment is bound by protein, it will be larger in size and its migration will therefore be retarded relative to that of the free fragment. For example, one method for a electrophoretic gel mobility shift assay can be (a) contacting in a mixture a nucleic acid binding protein with a non-radioactive or radioactive labeled nucleic acid molecule comprising a molecular probe under suitable conditions to promote specific binding interactions between the protein and the probe in forming a complex, wherein said probe is selected from the group consisting of dsDNA, ssDNA, and RNA; (b) electrophoresing the mixture; (c) transferring, using positive pressure blot transfer or capillary transfer, the complex to a membrane, wherein the membrane is positively charged nylon; and (d) detecting the complex bound to the membrane by detecting the non-radioactive or radioactive label in the complex.

The Biacore technology measures binding events on the sensor chip surface, so that the interactant attached to the surface determines the specificity of the analysis. Testing the specificity of an interaction involves simply analyzing whether different molecules can bind to the immobilized interactant. Binding gives an immediate change in the surface plasmon resonance (SPR) signal, so that it is directly apparent whether an interaction takes place or not. SPR-based biosensors monitor interactions by measuring the mass concentration of biomolecules close to a surface. The surface is made specific by attaching one of the interacting partners. Sample containing the other partner(s) flows over the surface: when molecules from the sample bind to the interactant attached to the surface, the local concentration changes and an SPR response is measured. The response is directly proportional to the mass of molecules that bind to the surface.

SPR arises when light is reflected under certain conditions from a conducting film at the interface between two media of different refractive index. In the Biacore technology, the media are the sample and the glass of the sensor chip, and the conducting film is a thin layer of gold on the chip surface. SPR causes a reduction in the intensity of reflected light at a specific angle of reflection. This angle varies with the refractive index close to the surface on the side opposite from the reflected light. When molecules in the sample bind to the sensor surface, the concentration and therefore the refractive index at the surface changes and an SPR response is detected. Plotting the response against time during the course of an interaction provides a quantitative measure of the progress of the interaction. The Biacore technology measures the angle of minimum reflected light intensity. The light is not absorbed by the sample: instead the light energy is dissipated through SPR in the gold film. SPR response values are expressed in resonance units (RU). One RU represents a change of 0.0001° in the angle of the intensity minimum. For most proteins, this is roughly equivalent to a change in concentration of about 1 pg/mm2 on the sensor surface. The exact conversion factor between RU and surface concentration depends on properties of the sensor surface and the nature of the molecule responsible for the concentration change.

There are a number of other assays that can determine whether an oligonucleotide or analogue thereof, peptide, polypeptide, oligosaccharide or small molecule can bind to the aptamer in a manner such that the interaction with the target is modified. For example, electrophoretic mobility shift assays (EMSAs), titration calorimetry, scintillation proximity assays, sedimentation equilibrium assays using analytical ultracentrifugation (see for eg. www.cores.utah.edu/interaction), fluorescence polarization assays, fluorescence anisotropy assays, fluorescence intensity assays, fluorescence resonance energy transfer (FRET) assays, nitrocellulose filter binding assays, ELISAs, ELONAs (see, for example, U.S. Pat. No. 5,789,163), RIAs, or equilibrium dialysis assays can be used to evaluate the ability of an agent to bind to a aptamer. Direct assays in which the interaction between the agent and the aptamer is directly determined can be performed, or competition or displacement assays in which the ability of the agent to displace the aptamer from its target can be performed (for example, see Green, Bell and Janjic, Biotechniques 30(5), 2001, p 1094 and U.S. Pat. No. 6,306,598). Once a candidate modulating agent is identified, its ability to modulate the activity of a aptamer for its target can be confirmed in a bioassay. Alternatively, if an agent is identified that can modulate the interaction of a aptamer with its target, such binding assays can be used to verify that the agent is interacting directly with the aptamer and can measure the affinity of said interaction.

In another embodiment, mass spectrometry can be used for the identification of an regulator that binds to a aptamer, the site(s) of interaction between the regulator and the aptamer, and the relative binding affinity of agents for the aptamer (see for example U.S. Pat. No. 6,329,146, Crooke et al). Such mass spectral methods can also be used for screening chemical mixtures or libraries, especially combinatorial libraries, for individual compounds that bind to a selected target aptamer that can be used in as modulators of the aptamer. Furthermore, mass spectral techniques can be used to screen multiple target aptamers simultaneously against, e.g. a combinatorial library of compounds. Moreover, mass spectral techniques can be used to identify interaction between a plurality of molecular species, especially “small” molecules and a molecular interaction site on a target aptamer.

In vivo or in vitro assays that evaluate the effectiveness of a regulator in modifying the interaction between a aptamer and a target are specific for the disorder being treated. There are ample standard assays for biological properties that are well known and can be used. Examples of biological assays are provided in the patents cited in this application that describe certain aptamers for specific applications.

The present invention also provides methods to identify the modulators of aptamers. Modulators can be identified in general, through binding assays, molecular modeling, or in vivo or in vitro assays that measure the modification of biological function. In one embodiment, the binding of a modulator to a nucleic acid is determined by a gel shift assay. In another embodiment, the binding of a modulator to a aptamer is determined by a Biacore assay.

In one embodiment, the modulator has the ability to substantially bind to a aptamer in solution at modulator concentrations of less than one (1.0) micromolar (uM), preferably less than 0.1 uM, and more preferably less than 0.01 uM. By “substantially” is meant that at least a 50 percent reduction in target biological activity is observed by modulation in the presence of the a target, and at 50% reduction is referred to herein as an IC₅₀ value.

### Pharmaceutical Compositions

The aptamers or modulators of the invention can be formulated into pharmaceutical compositions that can include a pharmaceutically acceptable carrier, diluent or excipient. The precise nature of the composition will depend, at least in part, on the nature of the aptamer and/or modulator, including any stabilizing modifications, and the route of administration. Generally, the aptamer or modulator is administered IV, IM, IP, SC, orally or topically, as appropriate.

Pharmaceutically useful compositions comprising an aptamer or modulator of the present invention can be formulated according to known methods such as by the admixture of a pharmaceutically acceptable carrier. Examples of such carriers and methods of formulation can be found in Remington's Pharmaceutical Sciences. To form a pharmaceutically acceptable composition suitable for effective administration, such compositions will contain an effective amount of the aptamer or modulator. Such compositions can contain admixtures of more than one compound.

In the methods of the present invention, the compounds can form the active ingredient, and are typically administered in admixture with suitable pharmaceutical diluents, excipients or carriers (collectively referred to herein as “carrier” materials) suitably selected with respect to the intended form of administration, that is, oral tablets, capsules, elixirs, syrup, suppositories, gels and the like, and consistent with conventional pharmaceutical practices.

For oral administration in the form of a tablet or capsule, the active drug component can be combined with an oral, non-toxic pharmaceutically acceptable inert carrier such as ethanol, glycerol, water and the like. Moreover, when desired or necessary, suitable binders, lubricants, disintegrating agents and coloring agents can also be incorporated into the mixture. Suitable binders include without limitation, starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include, without limitation, sodium oleate, sodium stearate, magnesium stearate, sodium benzoate, sodium acetate, sodium chloride and the like. Disintegrators include, without limitation, starch, methyl cellulose, agar, bentonite, xanthan gum and the like.

For liquid forms the active drug component can be combined in suitably flavored suspending or dispersing agents such as the synthetic and natural gums, for example, tragacanth, acacia, methyl-cellulose and the like. Other dispersing agents that can be employed include glycerin and the like. For parenteral administration, sterile suspensions and solutions are desired. Isotonic preparations that generally contain suitable preservatives are employed when intravenous administration is desired.

Topical preparations containing the active drug component can be admixed with a variety of carrier materials well known in the art, such as, e.g., alcohols, aloe vera gel, allantoin, glycerine, vitamin A and E oils, mineral oil, PPG2 mydstyl propionate, and the like, to form, e.g., alcoholic solutions, topical cleansers, cleansing creams, skin gels, skin lotions, and shampoos in cream or gel formulations.

The compounds of the present invention can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

The compounds of the present invention can also be coupled with soluble polymers as targetable drug carriers. Such polymers can include polyvinyl-pyrrolidone, pyran copolymer, polyhydroxypropylmethacryl-amidephenol, polyhydroxy-ethylaspartamidepbenol, or polyethyl-eneoxidepolylysine substituted with palmitoyl residues. Furthermore, the compounds of the present invention can be coupled (preferably via a covalent linkage) to a class of biodegradable polymers useful in achieving controlled release of a drug, for example, polyethylene glycol (PEG), polylactic acid, polyepsilon caprolactone, polyhydroxy butyric acid, polyorthoesters, polyacetals, polydihydro-pyrans, polycyanoacrylates and cross-linked or amphipathic block copolymers of hydrogels. Cholesterol and similar molecules can be linked to the aptamers to increase and prolong bioavailability.

The compounds can be administered directly (e.g., alone or in a liposomal formulation or complexed to a carrier (e.g., PEG)) (see for example, U.S. Pat. No. 6,147,204, U.S. Pat. No. 6,011,020). In one embodiment, a plurality of modulators can be associated with a single PEG molecule. The modulator can be to the same or different aptamer. In embodiments where there are multiple modulators to the same aptamer, there is an increase in avidity due to multiple binding interactions with the aptamer. In yet a further embodiment, a plurality of PEG molecules can be attached to each other. In this embodiment, one or more modulators to the same aptamer or different aptamers can be associated with each PEG molecule. This also results in an increase in avidity of each modulator to its target.

Lipophilic compounds and non-immunogenic high molecular weight compounds with which the modulators of the invention can be formulated for use in the present invention and can be prepared by any of the various techniques presently known in the art or subsequently developed. Typically, they are prepared from a phospholipid, for example, distearoyl phosphatidylcholine, and may include other materials such as neutral lipids, for example, cholesterol, and also surface modifiers such as positively charged (e.g., stearylamine or aminomannose or aminomannitol derivatives of cholesterol) or negatively charged (e.g., diacetyl phosphate, phosphatidyl glycerol) compounds. Multilamellar liposomes can be formed by the conventional technique, that is, by depositing a selected lipid on the inside wall of a suitable container or vessel by dissolving the lipid in an appropriate solvent, and then evaporating the solvent to leave a thin film on the inside of the vessel or by spray drying. An aqueous phase is then added to the vessel with a swirling or vortexing motion which results in the formation of MLVs. UVs can then be formed by homogenization, sonication or extrusion (through filters) of MLV's. In addition, UVs can be formed by detergent removal techniques. In certain embodiments of this invention, the complex comprises a liposome with a targeting aptamer(s) associated with the surface of the liposome and an encapsulated therapeutic or diagnostic agent. Preformed liposomes can be modified to associate with the aptamers. For example, a cationic liposome associates through electrostatic interactions with the nucleic acid. Alternatively, a nucleic acid attached to a lipophilic compound, such as cholesterol, can be added to preformed liposomes whereby the cholesterol becomes associated with the liposomal membrane. Alternatively, the nucleic acid can be associated with the liposome during the formulation of the liposome.

### Methods of Administration

Preferred modes of administration of the materials of the present invention to a mammalian host are parenteral, intravenous, intradermal, intra-articular, intra-synovial, intrathecal, intra-arterial, intracardiac, intramuscular, subcutaneous, intraorbital, intracapsular, intraspinal, intrasternal, topical, transdermal patch, via rectal, vaginal or urethral suppository, peritoneal, percutaneous, nasal spray, surgical implant, internal surgical paint, infusion pump or via catheter. In one embodiment, the agent and carrier are administered in a slow release formulation such as an implant, bolus, microparticle, microsphere, nanoparticle or nanosphere. For standard information on pharmaceutical formulations, see Ansel, et al., Pharmaceutical Dosage Forms and Drug Delivery Systems, Sixth Edition, Williams & Wilkins (1995).

The aptamers or modulators of the present invention can be administered parenterally by injection or by gradual infusion over time. Although the tissue to be treated can typically be accessed in the body by systemic administration and therefore most often treated by intravenous administration of therapeutic compositions, other tissues and delivery techniques are provided where there is a likelihood that the tissue targeted contains the target molecule. Thus, aptamers and modulators of the present invention are typically administered orally, topically to a vascular tissue, intravenously, intraperitoneally, intramuscularly, subcutaneously, intra-cavity, transdermally, and can be delivered by peristaltic techniques. As noted above, the pharmaceutical compositions can be provided to the individual by a variety of routes such orally, topically to a vascular tissue, intravenously, intraperitoneally, intramuscularly, subcutaneously, intra-cavity, transdermally, and can be delivered by peristaltic techniques. Representative, non-liming approaches for topical administration to a vascular tissue include (1) coating or impregnating a blood vessel tissue with a gel comprising a nucleic acid ligand, for delivery in vivo, e.g., by implanting the coated or impregnated vessel in place of a damaged or diseased vessel tissue segment that was removed or by-passed; (2) delivery via a catheter to a vessel in which delivery is desired; (3) pumping a nucleic acid ligand composition into a vessel that is to be implanted into a patient. Alternatively, the nucleic acid ligand can be introduced into cells by microinjection, or by liposome encapsulation. Advantageously, nucleic acid ligands of the present invention can be administered in a single daily dose, or the total daily dosage can be administered in several divided doses. Thereafter, the modulator is provided by any suitable means to alter the effect of the nucleic acid ligand by administration of the modulator.

The therapeutic compositions comprising modulator polypeptides of the present invention are conventionally administered intravenously, as by injection of a unit dose, for example. The term “unit dose” when used in reference to a therapeutic composition of the present invention refers to physically discrete units suitable as unitary dosage for the subject, each unit containing a predetermined quantity of active material calculated to produce the desired therapeutic effect in association with the required diluent; i.e., carrier or vehicle.

The compositions are administered in a manner compatible with the dosage formulation, and in a therapeutically effective amount as described herein. Suitable regimes for administration are variable, but are typified by an initial administration followed by repeated doses at one or more hour intervals by a subsequent injection or other administration. Alternatively, continuous intravenous infusion sufficient to maintain concentrations in the blood in the ranges specified for in vivo therapies are contemplated.

As used herein, the terms “pharmaceutically acceptable,” “physiologically tolerable,” and grammatical variations, thereof, as they refer to compositions, carriers, diluents and reagents, are used interchangeably and represent that the materials are capable of administration without substantial or debilitating toxic side effects.

Pharmaceutically useful compositions comprising a modulator of the present invention can be formulated according to known methods such as by the admixture of a pharmaceutically acceptable carrier. Examples of such carriers and methods of formulation can be found in Remington's Pharmaceutical Sciences. To form a pharmaceutically acceptable composition suitable for effective administration, such compositions will contain an effective amount of the aptamer. Such compositions can contain admixtures of more than one modulator.

### EXAMPLES

### Measures of Testing Coagulation

Standard measures of coagulation include the plasma-based prothrombin time (PT) and activated partial thromboplastin time (APTT) assays, both in plasma and whole blood, and the whole blood-based activated clotting time (ACT) assay. While the activators used to initiate coagulation in each of these assays are different, they share the common feature of clot formation as the endpoint for the assay. Importantly, in these in vitro assays, low levels of thrombin, ˜10-30 nM, are sufficient to produce enough fibrin to reach the endpoint. This level of thrombin represents conversion of only 3-5% of prothrombin to thrombin, and is consistent with the amount of thrombin generated during the initiation phase of the coagulation reaction (Butenas et al., 2003; Mann et al., 2003). Thus, these assays report largely on the initiation phase of the coagulation reaction, and do not fully reflect the impact of a deficiency in, or inhibition of, coagulation factors primarily involved in the propagation phase of coagulation.

The manner in which the standard clot-based assays reflect FIX/IXa activity is exemplified by their ability to detect or not detect abnormal coagulation measures in individuals with severe hemophilia A (a FVIII deficiency) or B (a FIX deficiency). A hallmark of hemophilia is the isolated prolongation of the APTT, as individuals with hemophilia have abnormal APTTs, but normal PTs (Bolton-Maggs and Pasi, 2003). The cell-based model of coagulation explains the paradox as to why individuals deficient in FVIII or FIX register normal PTs. The PT assay is initiated with supra-physiologic levels of tissue factor, enough to yield a clot in 11-15 seconds. Therefore, the high levels of tissue factor-FVIIa complex used to initiate the reaction rapidly produce FXa in amounts sufficient to yield enough thrombin to reach the clot endpoint, even in the absence of FVIII or FIX. Thus, even profound inhibition of FIX/FIXa activity is not expected to impact a PT assay, as the role of FIX in the initiation of coagulation is masked, or bypassed, in this assay. Thus, pharmacologic inhibitors of FIXa, such as the anti-FIXa aptamer RB006, are not expected to prolong PT values.

Both plasma or whole blood APTT assays are initiated with a charged particulate, such as celite or kaolin, a phospholipid surface, and calcium in sufficient quantities to yield a clot in ˜28-35 seconds. Individuals with hemophilia B (and A) register abnormal APTT values; however, the magnitude of the prolongation of APTT in these individuals is finite (i.e., yields a limited value), as the assay largely reports on the initiation phase of coagulation. There is not a tight correlation between the severity of an individual's hemophilia B and their APTT value, as the APTT is dependent upon other coagulation factors in addition to FIX. Therefore, a better framework for interpreting how pharmacologic inhibition of FIXa is expected to register in the APTT assay is the plasma FIX assay. The plasma FIX assay is a variation of the standard APTT method in which test plasma is diluted in buffer and mixed with FIX-deficient plasma prior to performing the APTT, such that the FIX level in the test plasma is the primary determinant of the clot time. This assay is typically used to determine the severity of hemophilia B (i.e., determine FIX levels) or to diagnose acquired inhibitors of FIX. The results of the FIX assay are interpreted by comparing the clot time of the test sample to a FIX-level standard curve, which is prepared by serial dilution of normal plasma in buffer prior to mixing with FIX-deficient plasma. Table 1 shows a typical FIX level standard curve performed with normal human plasma. [NOTE: Absolute APTT times in this assay are reagent-dependent.] As observed in Table 1, at levels of FIX that are 25% normal (i.e., reduced 75%), APTT clot times are increased 1.4-fold above baseline. At FIX levels ˜3% normal (i.e., reduced by 97%), APTT clot times are increased 2-fold above baseline, and at FIX levels <1% normal (i.e., reduced >99%), APTT clot times are increased 2.5 fold relative to baseline. Carriers of hemophilia B (i.e. ˜50% normal FIX levels) exhibit normal APTT values (Bolton-Maggs and Pasi, 2003), which is consistent with the data from the FIX level standard curve. Taken together, these observations indicate that a significant percentage of FIX activity must be inhibited before the APTT will be prolonged.

TABLE 1
FIX Activity Assay Standard Curve in Human Plasma

| % FIX Level | APTT Clot Time | Fold increase in Clot Time |
|---|---|---|
| 100* | 48.0 | 1.0 |
| 50 | 58.6 | 1.2 |
| 25 | 65.4 | 1.4 |
| 12.5 | 75.1 | 1.6 |
| 6.25 | 85.1 | 1.8 |
| 3.13 | 97.0 | 2.0 |
| 1.56 | 105.8 | 2.2 |
| 0.78 | 119.7 | 2.5 |

*100% FIX level represents a 1:5 dilution of normal pooled human plasma in buffer

Because ACT assays are used primarily in operating rooms and catheterization labs to monitor anticoagulation during procedures, little data exist as to how the ACT is impacted by reduced FIX/FIXa activity, as individuals with hemophilia are typically treated with factor replacement therapy (or a similar therapy) prior to undergoing such procedures. However, as the ACT is a clotting endpoint assay initiated with charged particulates, the effect of pharmacologic inhibition of FIXa in the ACT assay likely mirrors that observed in the APTT assay. That is, it is anticipated that prolongation of the ACT will not be observed until a substantial degree of FIXa inhibition is reached (>50%). Hence, analogous to the APTT assay, the magnitude of the prolongation of the ACT is likely to be modest as compared to the prolongation observed with unfractionated heparin. Finally, the assay is likely to saturate in response to FIXa inhibition. This similarity in the APTT and ACT response was demonstrated in monkeys treated with various doses of RB006 in the nonclinical toxicity studies.

### Effects of the REG1 Anticoagulation System on Measures of Coagulation

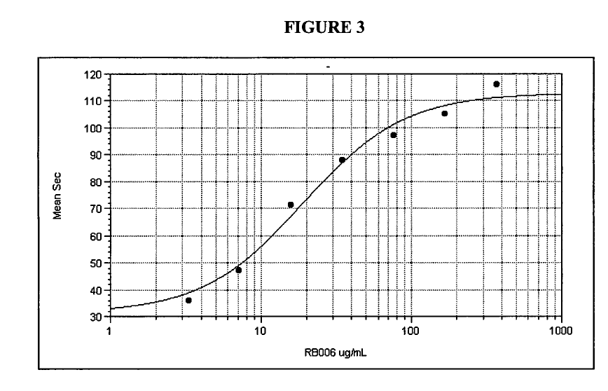

Previous data show that the anti-FIXa aptamers do not prolong PT, either in vitro or following IV administration to animals (Rusconi et al., 2004, Nat Biotechnol. 22(11): 1423-8; Rusconi et al., 2002, Nature 419(6902):90-4; Dyke, 2006, Circulation. 114(23):2490-7). As shown in FIG. 3, RB006 elicits a dose-dependent increase in the APTT in pooled normal human plasma in vitro. This data indicates that the RB006 APTT dose-response curve is most sensitive between 0 and 30-50 μg/mL, and then begins to plateau. These features including a rise phase and a plateau phase of the APTT dose-response curve are consistent in plasma from all species in which RB006 or prior anti-FIX aptamers exhibit cross-reactivity, including human, pig, mouse and monkey (Rusconi et al., 2004, Nat Biotechnol. 22(11):1423-8). The maximum APTT achieved in response to treatment of plasma in vitro with the anti-FIXa aptamer is dependent on the APTT reagent used and the species. Importantly, however, this maximum APTT is consistent with complete or near complete inhibition of FIXa activity. This is evidenced by the fact that the maximum APTT in response to the anti-FIXa aptamer is equivalent to the APTT in human plasma containing <1% normal FIX levels (but normal in all other clotting factor levels) and to the APTT in plasma from FIX-knockout mice Rusconi et al., 2004, Nat Biotechnol. 22(11):1423-8). Thus, the plateau of the APTT in response to RB006 likely reflects saturation of FIX/FIXa inhibition by the aptamer.

In addition, comparison of the data in FIG. 3 with the plasma FIX assay standard curve in Table 1 provides insight into the potency of RB006. The APTT increases ˜1.4 fold in response to RB006 at an RB006 concentration of ˜5 μg/mL, indicating this concentration of RB006 is sufficient to inhibit ˜75% plasma FIX activity. Furthermore based upon the plasma FIX assay, nearly 95% inhibition of plasma FIX (a 2.0-fold increase in APTT) is achieved at an RB006 concentration of 10 to 15 μg/mL.

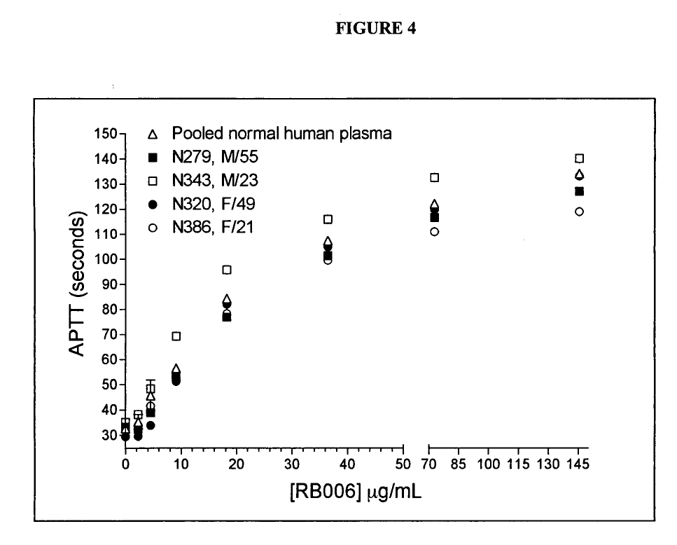

In vitro studies have been conducted to assess the individual variability of the anticoagulant effect of RB006 by measuring the RB006 concentration-dependent prolongation of the APTT in plasma from individuals. A comparison of the in vitro RB006 APTT dose-response curve in pooled normal human plasma versus plasma from individuals is shown in FIG. 4.

As shown in FIG. 4, the RB006 concentration-dependent increase in the APTT is very similar in the plasma from each of the individuals. Furthermore, the RB006 concentration-dependent increase in the APTT in the plasma from individuals is very similar to that in pooled normal human plasma (20 donors per pool). RB006 also prolongs the clotting time as measured in the ACT assay (Rusconi et al., 2004, Nat Biotechnol. 22(11):1423-8). However, interpretation of the change in ACT as a function of RB006 concentration is limited at this time due to the difficulty of performing in vitro dose-response studies with the ACT, as this assay requires fresh whole blood, and is time-sensitive.

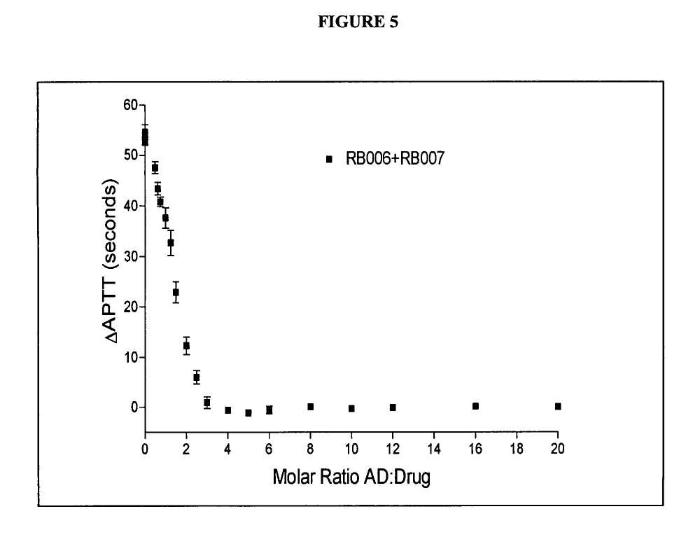

The neutralization of the anticoagulant activity of RB006 by the antidote RB007 has been measured in vitro using the APTT assay. As shown in FIG. 5, as the concentration of RB007 is increased relative to a fixed concentration of RB006 in pooled human plasma, the change in the APTT value returns to baseline levels, indicating complete neutralization of the anticoagulant activity of RB007. The minimum molar excess of RB007 required for complete RB006 neutralization in vitro in human plasma is approximately 3- to 4-fold (i.e., the molar ratio of the antidote relative to the oligonucleotide portion of the aptamer). This is consistent with the measured thermodynamic stability of the RB006-RB007 duplex (Tₘ of ˜90° C.).

The data presented in FIG. 5 also serve as the basis for the selection of the ratio of the dose of antidote RB007 relative to the drug RB006 used in the nonclinical safety pharmacology and toxicity studies and clinical trials. The minimum molar excess of RB007 relative to RB006 necessary to achieve complete neutralization of RB006 in vitro in human plasma is 3- to 4-fold. Given the difference in molecular weight between RB007 (5,269 Da, sodium salt) and RB006 (˜50,964 Da, sodium salt), this converts to a weight-to-weight ratio of 0.5:1 antidote:drug. As this is an in vitro result and therefore does not predict how the pharmacokinetics of either component will impact drug neutralization in vivo, the 0.5:1 weight ratio of antidote:drug reflects the minimum ratio of antidote that would be anticipated to effectively neutralize the drug. Therefore, a weight-to-weight ratio of 2:1 antidote:drug, a small multiple of the minimal effective dose ratio in vitro, was selected as a starting dose for nonclinical and clinical studies.

In summary, the anti-FIXa aptamer RB006 is a potent inhibitor of coagulation FIXa, capable of complete, or near complete, inhibition of FIXa activity in vitro. The anticoagulant activity of RB006 can be effectively monitored with APTT and ACT assays, as can the neutralization of aptamer activity by RB007. From in vitro studies, the relationship between the percentage FIX inhibition versus the change in APTT has been well defined for RB006. An appropriate molar ratio of antidote to aptamer sufficient to achieve complete inhibition of aptamer activity has also been defined from in vitro studies, which yielded the 2:1 mg/kg dose ratio of the antidote:aptamer chosen for the REG1 anticoagulation system.

### Nonclinical Pharmacology, Drug Disposition, and Toxicity

The pharmacologic activity of the REG1 anticoagulation system and its individual drug and antidote components (or less potent prototypes of the drug and antidote, referred to as RB002 and RB004 respectively) were demonstrated in vitro and in clinically relevant animal models.

The anticoagulant activity of the anti-FIXa aptamer was evaluated in systemic anticoagulant studies in pigs (Rusconi et al., 2004, Nat Biotechnol. 22(11):1423-8), in sheep cardiopulmonary bypass models, and in a safety pharmacology study in cynomolgus monkeys. The anti-thrombotic activity of the anti-FIXa aptamer was also demonstrated in a mouse arterial damage model (Rusconi et al., 2004, Nat Biotechnol. 22(11):1423-8). The drug neutralization activity of the antidote was demonstrated in vitro in human plasma (Rusconi et al., 2002, Nature 419(6902):90-4), in pig systemic anticoagulation models, in mouse models of surgical trauma (i.e., tail transection of highly anticoagulated animals) (Rusconi et al., 2004, Nat Biotechnol. 22(11):1423-8), in sheep cardiopulmonary bypass models, and in a safety pharmacology study in cynomolgus monkeys. In addition, the ability of the drug to be re-administered shortly after antidote neutralization of a prior drug dose was demonstrated in pig systemic anticoagulation studies.

Characterization of the pharmacokinetics of the REG1 anticoagulation system required a bioanalytical strategy that relied on novel methodology to quantify the levels of the aptamer, antidote and aptamer/antidote complex in plasma samples. These methods were applied to samples collected from the in vivo toxicity studies, which permitted determination of the pharmacokinetics of all three molecular entities under conditions of single and repeated dosing in monkeys and mice.

A thorough safety assessment of the REG1 anticoagulation system was conducted. The primary toxicity studies were performed in monkeys and mice under dosing conditions that simulated the intended use of the product in initial clinical trials (i.e., with sequential administration of aptamer followed 3 hours later by antidote administration). Small-to-large clinical multiples of each component were tested in the same dose ratio as intended for clinical use, and for both species the effects of the aptamer and antidote were tested separately. In both monkey studies, there were numerous treatment groups that received single doses of the aptamer, antidote or both test articles according to a schedule that mimicked the intended administration in initial clinical trials. Also, in the 14-day mouse study and in the single and repeated-dose monkey toxicity study, groups were included that were given repeated doses over a period of two weeks (14 daily doses for mice, and 7 doses, administered every other day for two weeks, for monkeys. Specialized endpoints were included in the toxicity studies to assess pharmacodynamic responses, exposure to REG1 components, and the class effects of oligonucleotides. The core toxicity studies were supplemented with safety pharmacology evaluation in monkeys (using radiotelemetry), a battery of genetic toxicity assays, and a blood compatibility study.

### Studies of Anticoagulant and Drug Neutralization Activity in Pigs

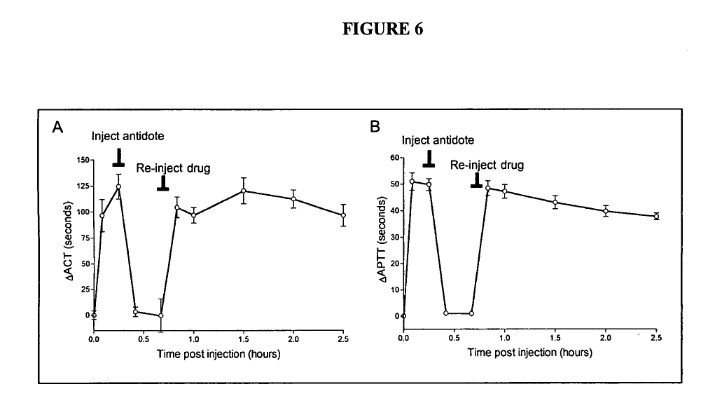

The ability to re-dose aptamer RB006 following antidote RB007 neutralization of an initial dose of the aptamer was evaluated in the porcine systemic anticoagulation model. In these studies, the second dose of the drug was administered 30 minutes following administration of the antidote. The 30-minute window between administration of the antidote and re-dosing with the aptamer was chosen to enable clear experimental demonstration of neutralization of the anticoagulant activity of the first aptamer dose. As shown in FIG. 6, the peak anticoagulant activity and time to peak anticoagulant activity of the second dose of the aptamer were essentially the same as with the initial aptamer dose, demonstrating that re-dosing with the aptamer following antidote-neutralization of the first aptamer dose is feasible. These data are in agreement with the observed pharmacokinetics of RB007 in both mice and monkeys, which indicate that RB007 possesses a very short plasma half-life (i.e., a few minutes) and does not accumulate to appreciable plasma concentrations even at substantially higher doses than used in this study. Given the half-life of the antidote, it is likely that the aptamer can be effectively re-administered at a shorter time interval than 30 minutes following antidote dosing.

Effectiveness of the REG1 Anticoagulation System in a Coronary Artery Bypass Graft (CABG) while on Cardiopulmonary Bypass in Sheep

REG1 can be used as an antidote-reversible anticoagulant in coronary revascularization procedures [coronary artery bypass graft (CABG) and percutaneous cardiac intervention (PCI)], as an antidote-reversible anticoagulant for use in patients, including humans, suffering from acute coronary syndromes, and as an anticoagulant for other indications in which it would be advantageous to employ an antidote-reversible agent for anticoagulant or antithrombotic therapy. The studies described herein are intended to define the range of doses of the anticoagulant component of REG1, RB006, necessary to maintain the patency of a cardiopulmonary bypass (CPB) circuit in an animal undergoing CABG surgery with CPB, and to define the corresponding dose of the antidote component of REG1, RB007, required to neutralize RB006 in this model.

RB006 (anti-coagulation agent) was administered intravenously to 10 sheep at the start of coronary artery bypass surgery. At the conclusion of surgery, the RB007 (RB006 neutralizing agent) was given intravenously to reverse the effects of RB006. After 28±3 days all animals were euthanized.

Representative samples of right and left kidneys, liver, lung, and the entire brain were collected. Hearts were flushed with lactated Ringer's solution or normal saline until cleared of blood and pressure-perfusion fixed at ˜100 mmHg with 10% neutral buffered formalin (NBF) for a minimum of 6 hours. Upon complete fixation, the hearts were placed in 10% NBF. Representative tissue samples collected during necropsy were immersion fixed with 10% NBF.

The hearts were transversely sectioned approximately every 1 cm (in breadloaf fashion) and examined for abnormalities. Ten sections were collected from each heart and processed in paraffin. Three of the ten sections included: LCX anastomosis, aortic anastomosis, and mid-graft. The remaining seven sections included: right atrial wall, left atrial wall, interatrial septum, right ventricular free wall, left ventricular free wall, interventricular septum, and apex. All paraffin blocks containing myocardial tissue were sectioned twice, once for staining with hematoxylin and eosin (H&E) and once for staining with Masson's Verhoeff Elastin (MVE). The samples of kidneys, liver, lung, and brain were embedded in paraffin and sectioned as follows: one section from each kidney, one section from liver, one section from lung, and one section from each of the four samples of brain tissue, for a total of eight sections. All resulting slides were stained with H&E.

The macroscopic observations and histologic correlates for this study indicate that most of the lesions were either related to the surgical procedure (e.g. adhesions) or euthanasia (e.g. foam in trachea and bronchi). Adhesions are a common sequela for this type of procedure and were not considered excessive in this study.

There was a small, minimally attached thrombus at the aortic anastomosis in one animal. The thrombus did not appear to obstruct blood flow into the graft. There were no specific microscopic correlates for this observation. The microscopic findings at the anastomosis site were similar in type and magnitude to other study animals in both groups. With one exception, there was no macroscopic evidence of thrombosis or occlusion within any portion of the coronary artery bypass in any study animal. Occasional thrombus formation is not uncommon in this model; hence, a relationship to RB006 administration is considered doubtful.

### Pharmacodynamic Activity of the REG1 Anticoagulation System in Cynomolgus Monkeys

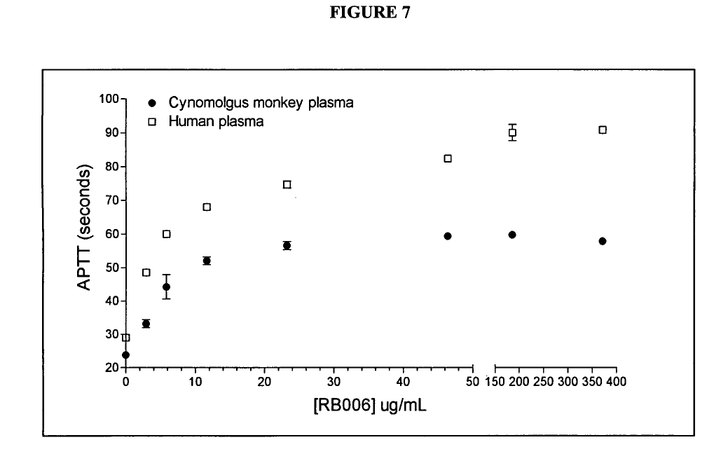

The in vitro anticoagulant activity of RB006 in plasma from cynomolgus monkeys is reflected by concentration-dependent prolongation of time-to-clot in the APTT assay. As can be seen in FIG. 7, the RB006 APTT dose-response curve is most sensitive between 0 and 50 μg/mL, and then plateaus, as has been seen with other species. The monkey and human dose-response curves are similar, except that the range of response is greater in humans. In human plasma, there is a concentration-dependent prolongation of the APTT up to approximately 200 μg/mL, whereas in monkey plasma, the concentration-response curve reaches a plateau at approximately 50 μg/mL. The plateau of the human plasma curve occurs at an APTT value equivalent to that observed in human plasma containing <1% plasma FIX activity, and is likely due to saturation of the target, FIXa. Plasma FIX assays were performed to aid in interpretation of the RB006 APTT dose-response curve in monkey plasma. As shown in Table 2, the APTT in monkey plasma is sensitive to the FIX level. However, the magnitude of the response to reduction in the FIX level is modest. A 75% reduction in the FIX level results in a 1.4-fold increase in the APTT, a >95% reduction in the FIX level results in a doubling of the APTT, and a 99.9% reduction in the plasma FIX level yields a 2.5-fold increase in the APTT.

TABLE 2
FIX Activity Assay Standard Curve
in Cynomolgus Monkey Plasma

| % FIX Level | APTT Clot Time | Fold increase in Clot Time |
|---|---|---|
| 100* | 35.1 | 1.0 |
| 50 | 41.9 | 1.2 |
| 25 | 49.4 | 1.4 |
| 12.5 | 55.9 | 1.6 |
| 6.25 | 62.2 | 1.8 |
| 3.13 | 68.0 | 1.9 |
| 1.56 | 74.7 | 2.1 |
| 0.78 | 77.7 | 2.2 |
| 0.39 | 83.8 | 2.4 |
| 0.098 | 88.1 | 2.5 |

*100% FIX level represents a 1:5 dilution of normal pooled cynomolgus plasma in buffer. Human FIX-deficient plasma (George King Biomedical) was used as the source of FIX-deficient plasma.

Comparison of the data in FIG. 7 to the data presented in Table 2 indicates that ˜6 μg/mL RB006 is required to inhibit approximately 90% of plasma FIX activity in monkeys (i.e., this concentration yields a 1.6-fold increase in the APTT), and that >95% inhibition of plasma FIX activity occurs at RB006 concentrations of 10-12 μg/mL. The in vitro RB006 monkey APTT dose-response curve plateaus at approximately a 2.5-fold increase over baseline (baseline ˜24 seconds, maximum APTT ˜60 seconds), which is consistent with the magnitude of the increase in the APTT observed in the monkey plasma FIX assay at <0.1% normal FIX levels (see Table 2). Therefore, the plateau in the RB006 APTT dose-response curve likely represents saturation of the target in monkey plasma (i.e., complete inhibition of FIX activity). In conclusion, the % FIX inhibition versus plasma RB006 concentration in vitro in monkey plasma is generally similar to that observed in vitro in human plasma, with the key differences being that the RB006 concentration range between the baseline and the maximum APTT is larger in humans, and the rise in the dose response is more gradual in human plasma than it is in monkey plasma.

### In Vivo Activity of RB006 and RB007 in Cynomolgus Monkeys

The relationship between the anticoagulant properties of RB006 and the RB006/RB007 complex and the plasma levels of these compounds was evaluated in the monkey safety pharmacology study REG1-TOX001. Briefly, 12 monkeys were assigned to three treatment groups. Group 1 received the anti-FIXa aptamer RB006, Group 2 received the antidote to RB006, RB007, and Group 3 was treated with the REG1 anticoagulation system, i.e., RB006 followed by RB007 (three hours later). Doses were escalated through two quantities of test articles, with the first dose occurring on Day 4 of the study and the second dose occurring on Day 13. To better understand the dose-response to RB006, the four monkeys assigned to Group 1 (RB006, aptamer alone) were subdivided into two groups at Day 13, with two animals receiving a low dose (Group 1a, 5 mg/kg RB006) and two animals receiving a high dose (Group 1b, 30 mg/kg RB006).

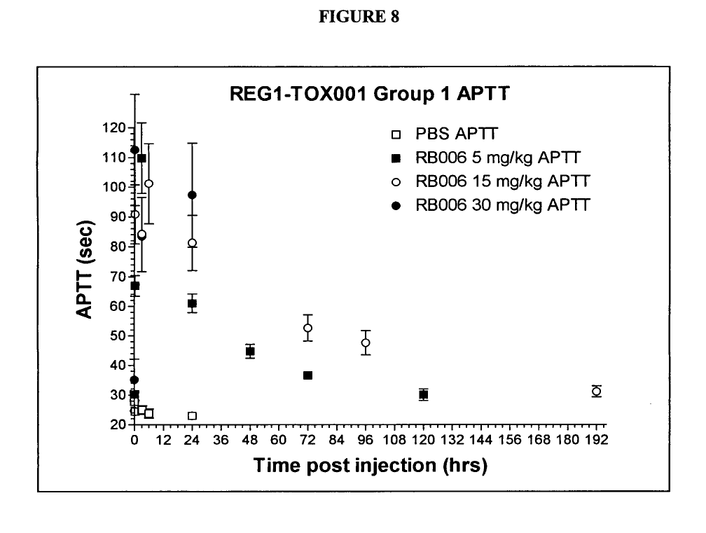

As shown in FIG. 8, administration of RB006 at doses ranging from 5 to 30 mg/kg resulted in a profound level of anticoagulation in the monkeys. The mean APTT at each dose level exceeded 60 seconds from 0.25 to 24 hours following RB006 administration, which is equivalent to <0.1% normal plasma FIX levels in the monkey. There is a dose-dependent increase in APTT in response to RB006 administration.

However, the dose-response is not immediately evident due to the fact that, up to the 6-hour time point following RB006 administration, the RB006 plasma level exceeded the concentration at which the in vitro APTT dose-response curve approaches a plateau (˜40-50 μg/mL; see Table 3 and FIG. 7). At times beyond 6 hours after RB006 administration, as the RB006 concentration decreases below this level, the dose-response is more apparent. APTT was followed until it returned to baseline in monkeys receiving 5 and 15 mg/kg doses of RB006. Mean APTT returned to baseline by 120 hours at the 5-mg/kg dose level and 192 hours at the 15-mg/kg dose level, consistent with both the in vitro APTT dose-response curve (FIG. 7) and the observed half-life of approximately 12 hours for RB006 in monkeys (see Table 3). The whole-blood activated clotting time (ACT) data mirrored the APTT data (data not shown).

Toxicokinetic data were collected at several time points over the first 24 hours after RB006 administration using a dual oligo hybridization ELISA assay. As shown in Table 3, the concentration of RB006 increased as a function of the dose administered, and the half-life of RB006 was in the 12-hour range. Consistent with the data presented in FIG. 8, comparison of the plasma levels of RB006 (Table 3) with the in vitro dose-response curve shown in FIG. 7 indicated the animals were profoundly anticoagulated throughout the first 24 hours post RB006 administration at all dose levels. These dose levels are well above the proposed clinical range. There is an excellent correspondence between the mean RB006 concentration 24 hours post administration in the Group 1a animals and the mean APTT of these animals. The mean RB006 concentration of the animals treated with 5 mg/kg RB006 at 24 hours was 15.9 μg/mL and the mean APTT was 61.1 seconds. This compares very favorably to the expected result based upon the in vitro RB006 dose-response curve in monkeys (see FIG. 7). Therefore, this study confirms the usefulness of the APTT to monitor the level of anticoagulation in monkeys treated with RB006, and the data support the use of the APTT to monitor the anticoagulation state of humans receiving RB006 in initial clinical studies.

TABLE 3
Group 1 REG1-TOX001 RB006 Plasma Levels (μg/mL)

| | Group 1 Dose Levels (animals/dose level) | | |
|---|---|---|---|
| Time Post | 5 mg/kg | 15 mg/kg | 30 mg/kg |
| Injection (hours) | (n = 2)* | (n = 4) | (n = 2)* |
| Pre-dose | 0.2 | <0.04 | 0.2 |
| 0.25 | 59.8 | 179.8 ± 28.9 | 465.5 |
| 3 | 66.6 | 145.6 ± 32.5 | 328.9 |
| 6 | 42.1 | 101.5 ± 13.4 | 275.3 |
| 24 | 15.9 | 51.1 ± 11.2 | 164.6 |

*For Day 13 dosing, animals were split into Group 1a (5 mg/kg) and 1b (30 mg/kg). For these dose levels, the average plasma level for the two animals per dose level is reported. The RB006 present in Group 1a and 1b animals at the pre-dose time point is residual RB006 from the 15-mg/kg dose at Day 4. The LLOQ of the assay is <0.04 μg/mL.

In the Group 2 animals treated with the antidote RB007 only, mean APTT and ACT were not affected by RB007 administration at either dose level tested (30 and 60 mg/kg). Toxicokinetic data were collected at several time points over the first 24 hours after RB007 administration using a dual oligo hybridization ELISA assay. As shown in Table 4, low, but measurable levels of the antidote were present in plasma from animals receiving RB007 at 0.25 hours after injection of 30 mg/kg on Day 4 or 60 mg/kg on Day 13. These levels were highly variable, but were generally dose-dependent. The post-dosing level of the antidote was very low by comparison to the concentration of the aptamer (in Group 1) following IV injection. Thus, it is clear that the antidote has a very short half-life in plasma when administered alone, and is largely cleared from circulation by 15 minutes following injection.

TABLE 4
Group 2 REG1-TOX001 RB007 Plasma Levels (μg/mL)

| | |
|---|---|
| Time Post | |
| RB007 Injection | Group 2 Dose Levels (4 animals/dose) |

| (hours) | 30 mg/kg | 60 mg/kg |
|---|---|---|
| Pre-dose | <0.01 | <0.01 |
| 3.25 | 0.4 ± 0.1 | 0.6 ± 0.5 |
| 6 | 0.02 ± 0.01* | <0.02*** |
| 24 | 0.01 ± 0.01** | <0.01*** |

*1 animal at < LLOQ of 0.01 included in calculations
**3 animals at < LLOQ of 0.01 included in calculations
***Average of LLOQs

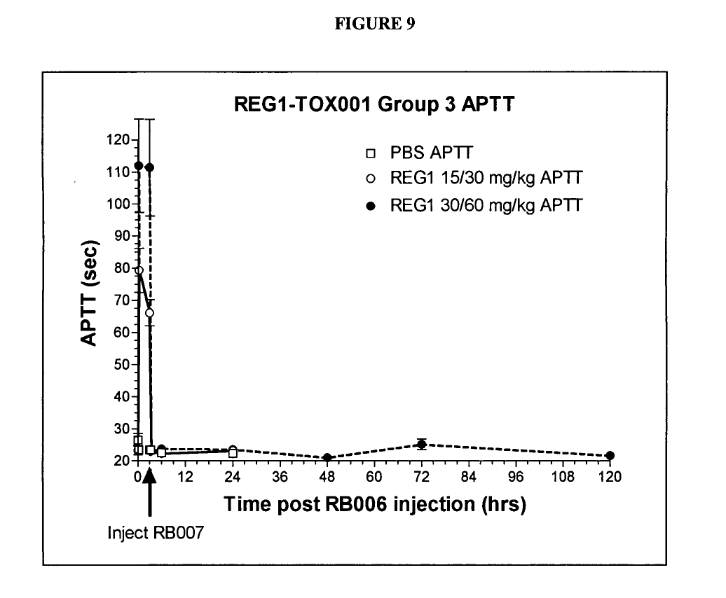

The APTT data from animals treated with RB006 followed by RB007 3 hours later (Group 3) are shown in FIG. 9. In agreement with the data from animals treated with RB006 only, administration of RB006 at these dose levels resulted in a profound level of anticoagulation, with the mean APTT's at 0.25 and 3 hours post administration consistent with essentially complete FIX inhibition at both dose levels. Subsequent administration of RB007 rapidly and completely neutralized the anticoagulant effects of RB006 in the monkey, with the mean APTT returning to baseline within 15 minutes following RB007 administration (the first time point taken) at both RB006/RB007 dose levels tested. In the Group 3 animals treated with 30/60 mg/kg RB006/RB007, the APTT was followed for 5 days post RB006 administration. APTT data collected over this time frame indicate the anticoagulant effects of RB006 were durably neutralized, with no evidence of rebound anticoagulation over 120 hours, or approximately 10 half-lives of RB006 in the monkey (FIG. 9). The durability of the neutralization of the anticoagulant activity of RB006 by the antidote RB007 is entirely consistent with the observed thermodynamic stability of this drug-antidote complex.

Toxicokinetic data were collected for 24 hours following RB006 administration in the Group 3 animals (Table 5). For Group 3 animals, both free RB006 (i.e., RB006 not bound by RB007) and complexed RB006 (i.e., RB006 bound by RB007) plasma concentrations were measured. Consistent with the APTT data presented in FIG. 9, the mean plasma concentrations of RB006 at 0.25 and 3 hours after administration were quite high. Within 15 minutes of RB007 administration, the mean concentration of free RB006 decreased 5,000-10,000 fold, to levels below the Lower Limit of Quantitation (LLOQ) of the assay employed. Concomitant with the decrease in free RB006 levels, the mean plasma concentration of complexed RB006 increased from below the LLOQ of the assay to ˜125 to 220 μg/mL at the 15/30 and 30/60 mg/kg dose levels respectively, indicating the rapid decrease in free RB006 concentrations was due to binding of RB007 to RB006. The concentration of free RB006 remained below the LLOQ of the assay as long as 3 hours after RB007 administration, consistent with the APTT results. At 21 hours after RB007 administration (24 hours after RB006 administration), very low levels of RB006 were detectable in several animals (mean of only 0.17 μg/mL or lower). However, these levels of RB006 are too low to exert a measurable anticoagulant effect, consistent with the absence of APTT prolongation at 24 hours and longer in animals treated with the REG1 anticoagulation system.

TABLE 5
Group 3 REG1-TOX001 Free and Complexed RB006 Plasma Levels (μg/mL)

| Time Post | Group 3 Dose Levels | | | |
|---|---|---|---|---|
| RB006 | 15/30 mg/kg RB006 + RB007 | | 30/60 mg/kg RB006 + RB007 | |
| Injection | Free | Complexed | Free | Complexed |
| (hours) | RB006 | RB006 | RB006 | RB006 |
| Pre-dose | <0.04 | ND | 0.05 ± 0.01 | ND |
| 0.25 | 280.2 ± 64.3 | ND | 467.6 ± 67 | ND |
| 3.0 | 214.6 ± 31.8 | <0.04 | 488.4 ± 68.6 | <0.04 |
| 3.25 | <0.04 | 125.1 ± 7.9 | <0.04 | 218.2 ± 27.2 |
| 6 | <0.04 | 98.7 ± 20.5 | <0.04 | 184.8 ± 28.9 |
| 24 | 0.14 ± 0.08* | 8.3 ± 4.5 | <0.04 ± 0.01** | 22.3 ± 12 |

*1 animal at < LLOQ of 0.04 μg/mL included in calculations
**3 animals at < LLOQ of 0.04 μg/mL included in calculations
RB007 administered at t = 3 hrs immediately after 3 hr blood draw.
(ND) Not determined.

### Summary of Nonclinical Pharmacology Studies in Monkeys

The studies presented demonstrate that RB006 is a potent anticoagulant in monkeys, capable of achieving essentially complete inhibition of FIX activity for 24 hours or longer following a single bolus IV injection of the drug at supra-clinical doses. Comparison of in vitro studies of the anticoagulant activity of RB006 in monkeys with the APTT and toxicokinetic data from this safety pharmacology study demonstrates a good correspondence between the expected and observed prolongation of the APTT versus the plasma RB006 concentration. Therefore, the APTT assay will serve as a useful tool to monitor anticoagulation induced by RB006 administration. The similarity between the in vitro human and monkey RB006-APTT dose-response curves suggests that the data derived from this monkey study (REG1-TOX001), as well as the large general toxicity study conducted in monkeys (REG1-TOX003) will serve as a useful guide in predicting the human response to administration of RB006. Finally, the APTT and toxicokinetic data from REG1-TOX001 demonstrate that RB007 is a very effective antidote for RB006. Within 15 minutes following bolus IV administration of RB007 in RB006-treated animals, mean APTT times returned to pre-RB006 treatment levels and remained at this baseline level for the entire monitoring period (up to 120 hours). The observed neutralization of the RB006 anticoagulant activity by RB007 was fully supported by toxicokinetic data, and is consistent with the measured thermodynamic stability of the RB006-RB007 complex. Toxicokinetic studies demonstrated that free RB006 levels decreased to below the LLOQ of the assay within 15 minutes post RB007 administration, concomitant with a significant rise in the concentration of complexed RB006, and without an appreciable increase in free RB006 levels for the duration of the toxicokinetic analysis (24 hours post RB006 administration). Therefore, the data obtained in monkey studies demonstrated that the REG1 anticoagulation system behaves as intended with respect to achieving stable, durable and monitorable anticoagulation from a single IV injection of the aptamer, followed by rapid, complete, and durable neutralization of aptamer activity upon IV bolus injection of the antidote. This performance of the REG1 anticoagulation system was achieved at low to high multiples of the intended clinical dose range (i.e., appropriate doses for toxicity studies), but without adverse effects on the animals.

### REG1 Toxicokinetics

Bioanalytical methods were developed and validated to enable quantification of the concentrations of free aptamer (RB006), free antidote (RB007) and aptamer/antidote (RB006/RB007) complex in plasma from monkeys and mice. These methods were applied to analysis of samples collected from the safety pharmacology study in monkeys (Study No. REG1-TOX001), the 14-day study in mice (Study No. REG1-TOX002), and the single/repeat-dose study in monkeys (Study No. REG1-TOX003). For all three studies, separate groups of animals were included that received either the aptamer alone, or the antidote alone, or the aptamer followed 3 hours later by the antidote. Multiple dose levels of each treatment condition were tested in all of the studies, and two of these studies (the 14-day study in mice and the single/repeat-dose study in monkeys) also employed repeated administration of the test articles. The dose levels of the aptamer tested in these studies ranged from 0.25 to 45 mg/kg in monkeys and 2.5 to 22.5 mg/kg in mice. The doses of the antidote tested were twice those of the aptamer (i.e., up to 90 mg/kg in monkeys and 45 mg/kg in mice). This ratio is analogous to that intended for use in clinical trials.

For all three studies, the toxicokinetic results were similar with respect to documenting the following properties of the REG1 anticoagulation system:
The plasma concentrations of the aptamer following intravenous injection were dose-proportional over a broad dose range, with a modest degree of inter-animal variation. No gender differences were apparent in either monkeys or mice.
The clearance of the aptamer from plasma was relatively slow (i.e., the estimated half-life was at least 12 hours in monkeys and ˜8 hours in mice). This slow clearance was expected based on the PEGylated structure of the aptamer and is consistent with literature reports on the pharmacokinetics of other PEGylated oligonucleotides. The minimal clearance of the aptamer, in combination with its high factor IX inhibitory potency, provided for a relatively stable degree of anticoagulation over a 6-hour period, based on measurement of pharmacodynamic markers, i.e., activated partial thromboplastin time and activated clotting time. This profile is a desirable property of the aptamer component of the REG1 anticoagulation system.
Intravenous injection of the antidote alone (without prior treatment with aptamer) yielded very low levels in plasma, even at the first sampling time following injection (10-15 minutes). The antidote levels measured at these early times were orders of magnitude lower than those of the aptamer (i.e., as compared to the aptamer levels in those groups that had received aptamer alone) despite the fact that the antidote dose levels were twice as high. Collectively, the data for the antidote indicate that it has a very short half-life in plasma when given alone. No accumulation of the antidote in plasma occurred when it was administered at a relatively high dose level (30 mg/kg) to monkeys every other day for 7 doses (14 days).
For the groups that received aptamer followed 3 hours later by the antidote (i.e., the complete REG1 anticoagulation system), the concentration of free aptamer was sharply reduced within minutes following antidote administration to below or slightly above the limits of quantification (using a highly sensitive hybridization-type assay), indicating complete binding of the circulating aptamer by the antidote. As was seen with the antidote-alone treatment, there were very low levels of free antidote under these conditions. The binding of the aptamer by the antidote was associated with virtually complete neutralization of aptamer activity (i.e., normalization of coagulation parameters), consistent with the intended performance of the REG1 anticoagulation system.
Concurrent with elimination of free aptamer, the aptamer/antidote complex was detected in plasma at levels consistent with the complete binding of aptamer by the antidote. The complex was eliminated from plasma at a rate slightly faster than that of the free aptamer (i.e., by comparison to the rate of aptamer clearance in groups treated with aptamer only) but at a much lower rate than free antidote, as would be expected from the presence of the polyethylene glycol moiety within the complex (derived from the aptamer). Extensive elimination of the aptamer/antidote complex from plasma was evident within 21 hours following antidote dosing. With repeated administration of the aptamer and antidote (the REG1 coagulation system) to monkeys every day for two weeks, there was no accumulation of the complex in the blood or the free aptamer, no change in aptamer pharmacokinetics (i.e., during the period prior to antidote dosing), and no evidence of cumulative anticoagulation exerted by the aptamer.
The only difference between the pharmacokinetics in mice and monkeys was the moderately longer half-life of the aptamer in monkeys (at least 12 hours, compared to ˜8 hours in mice).

### Clinical Use of REG1 in Humans

In choosing which method of anticoagulation to use for an individual patient or patient-population, clinicians weigh the characteristics of various pharmacologic strategies. Keeping in mind that the major adverse effect of anticoagulation is bleeding (i.e., exaggerated pharmacology), for acute-care indications the ideal anticoagulant would be 1) deliverable intravenously or subcutaneously, 2) immediately therapeutic, 3) easily dosed so as not to require frequent monitoring, and most importantly, 4) immediately and predictably reversible. The REG1 anticoagulation system has been developed in response to this unmet medical need for an effective, safe and rapidly reversible anticoagulant.

REG1 can be used in a number of clinical settings for the treatment of humans, and other animals, in need of such treatment. For example, REG1 can be used in coronary and peripheral revascularization procedures associated with artery disease and occlusions as an antidote-reversible anticoagulant. Specially, REG1 can be used as an antidote-reversible anticoagulant in coronary revascularization procedures (coronary artery bypass graft (CABG) and percutaneous cardiac intervention (PCI)), as an antidote-reversible anticoagulant for use in patients suffering from acute coronary syndromes, and as an anticoagulant for other indications in which it would be advantageous to employ an antidote-reversible agent for anticoagulant or antithrombotic therapy. Disorders and procedures for which the methods of the invention may be used include, but are not limited to, peripheral vessel graft procedures, including those associated with the iliac, carotid, brachial, aorta, renal, mesenteric, femoral, popliteal, tibial, and peritoneal vessels; the prevention of deep vein thrombosis; the prevention of pulmonary embolism following orthopedic surgery or in patients with cancer; the prevention of atrial fibrillation; the prevention of thrombotic stroke; and in indications requiring extracorporeal circulation of blood including but not limited to hemodialysis and extracorporeal membrane oxygenation. Additional examples of potential disorders and procedures for which the methods of the invention can be used include, but are not limited to, patients undergoing intracardiac surgery on cardiopulmonary bypass; patients with intracardiac clot formation or peripheral embolization; and patients that are in other hypercoagulable states. The methods of the invention may also be useful for prevention of DVT and pulmonary embolization on immobilized patients and for maintenance of potency of indwelling intravenous catheters and arterial or in venous lines

The range of doses of the anticoagulant component of REG1, RB006, will be dependant upon the indication. For example, the RB006 dose can be in humans from about 0.1 mg/kg to about 10 mg/kg. In certain indications, the dose range will be about from 0.5 mg/kg to about 9 mg/kg, from about 0.75 mg/kg to about 8 mg/kg, from about 1 mg/kg to about 7 mg/kg, from about 1.5 mg/kg to about 6.0 mg/kg, from about 2.0 mg/kg to about 5.0 mg/kg, from about 2.5 mg/kg to about 4.0 mg/kg. In certain indications, the drug component will be administered at a dose necessary to maintain the patency of the procedure. In certain indications, RB006 will be administered alone, without subsequent administration of a neutralizing antidote.

The corresponding dose of the antidote component of REG1, RB007, required to neutralize or partially neutralize RB006 is dependent upon the amount of RB006 administered. The antidote dose can range, in a antidote:drug weight ratio (mgs of antidote:mgs of drug), from about 0.1:1 to about 20:1, from about 0.25:1 to about 15:1, from about 0.5:1 to about 12:1, from about 0.75 to about 10:1, from about 1:1 to about 9:1, from about 1.5:1 to about 8:1, from about 2:1 to about 7.5:1, from about 2.5:1 to about 6:1, from about 3:1 to about 5:1.

The most important property of the REG1 anticoagulation system that fosters confidence in its safe clinical application is the well-established capacity for the antidote to predictably reverse the pharmacologic activity of the aptamer in a dose dependent manner.

### Evaluation of the REG1 Anticoagulation System in Humans

This study was the first time the REG1 anticoagulation system was evaluated in humans. Single intravenous (IV) dose-escalation studies of the REG1 anticoagulation system was performed in healthy human volunteers. Subjects in this study were randomly assigned to study article or placebo in one of three arms at one of four (4) different dose levels. In each arm at each dose level, subjects were randomized 7:1 to treatment vs. placebo, with subjects receiving REG1 or placebo. Sodium Chloride Injection 0.9% USP were used for all placebo injections. Subjects were randomized to receive REG1 or placebo at each dose level.

In order to minimize the risks to and maximize the safety of the subjects enrolled in this study, three arms were designated in the following order:
Arm 1: placebo drug followed by active RB007 antidote component OR placebo drug followed by placebo antidote
Arm 2: active RB006 drug followed by active RB007 component OR placebo drug followed by placebo antidote
Arm 3: active RB006 drug followed by placebo antidote OR placebo drug followed by placebo antidote

Arm 1 evaluated the antidote component of the REG1 anticoagulation system (RB007). Each subject in this arm received an injection of placebo at time 0 (ie. The time at which the first bolus injection is administered). Three (3) hours later, the subjects received an intravenous injection of the active antidote component (RB007), while one (1) subject received placebo.

Arm 2 evaluated the combination of the active drug component of the REG1 anticoagulation system (RB006) followed by the active antidote component of the REG1 anticoagulation system (RB007). The subjects in this arm received an injection of active drug component (RB006) at time 0, and one (1) received placebo. Three (3) hours later, the subjects who received active drug component received an injection of active antidote component (RB007), while the one (1) subject who received placebo in place of drug component received placebo in place of antidote.

Arm 3 evaluated the active drug component of the REG1 anticoagulation system (RB006). The subjects in this arm received an injection of active drug (RB006) at time 0 and one (1) received placebo in place of antidote. Three (3) hours later all of the subjects received placebo in place of antidote

The active study drug component (RB006) was administered at four (4) dose levels: (1) Low Dose (15 mg RB006); (2) Low Intermediate Dose (30 mg RB006); (3) High Intermediate Dose (60 mg RB006); and (4) High Dose (90 mg RB006). The starting dose and subsequent escalations were chosen to target maximum plasma concentrations that define three (3) key aspects of the in vitro APTT dose response curve for RB006 in pooled normal human plasma: a low dose targeting a maximum plasma concentration at which the APTT begins to rise in the RB006 in vitro dose response curve (˜4 μg/mL); two (2) intermediate doses targeting plasma concentrations that bracket the IC50 of the in vitro RB006 APTT dose response curve (˜8-16 μg/mL); and a high dose targeting a plasma concentration at which the in vitro RB006 APTT dose response curve begins to plateau (˜25 μg/mL).

The active study antidote component (RB007) was administered at four (4) corresponding dose levels equivalent to twice the drug (RB006) dose level on a mg/kg basis: (1) Low Dose (30 mg RB007); (2) Low Intermediate Dose (60 mg RB007); (3) High Intermediate Dose (120 mg RB007); and (4) High Dose (180 mg RB007). Table 6 outlines doses in each Arm for this Phase 1A study.

Study drug component (RB006), study antidote component (RB007), and their respective placebos were each given as an injection over a period of one (1) minute. The REG1 study drug component or placebo was given at time 0 and the antidote component or placebo was given at three (3) hours.

TABLE 6
Phase 1a Doses Planned for the Three Treatment Arms

| | | Arm 2: | | |
|---|---|---|---|---|
| | Arm 1: | Drug (RB006), | | Arm 3: |
| | Placebo + | mg + | | Drug (RB006), |
| | Antidote | Antidote | | mg + |
| Group | (RB007), mg | (RB007), mg | | Placebo |
| Dose Level 1: | 30 | 15 | 30 | 15 |
| Low Dose | | | | |
| Dose Level 2: | 60 | 30 | 60 | 30 |
| Low | | | | |
| Intermediate | | | | |
| Dose | | | | |
| Dose Level 3: | 120 | 60 | 120 | 60 |
| High | | | | |
| Intermediate | | | | |
| Dose | | | | |
| Dose Level 4: | 180 | 90 | 180 | 90 |
| High Dose | | | | |

REG1 was evaluated in healthy volunteers to determine the safety profile and describe the PK and PD responses of the REG1 anticoagulation system. This study was the first time an anticoagulation system utilizing an aptamer and an oligonucleotide antidote to the aptamer was administered to a human. The results indicate that a dose-response of APTT was seen following bolus IV injection of drug, with a rapid and sustained return to baseline APTT following antidote bolus IV injection. ACT followed a similar pattern as the APTT. PT remained unchanged compared to baseline.

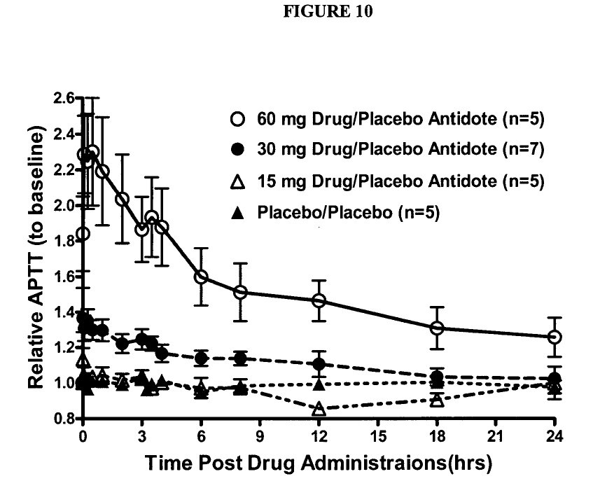

Subjects were administered RB006 or 0.9% normal saline as an intravenous bolus injection at time zero, and the anticoagulant effect of the treatment was assessed over time by measurement of the plasma APTT (FIG. 10). APTT values for each treatment group are expressed as the mean±SEM of the Relative APTT. The Relative APTT is the APTT value for an individual subject at a given sample time divided by the pre-RB006 administration baseline APTT value for that subject. A value of 1 indicates no response to RB006 and a value >1 indicates an anticoagulant effect. A clear dose-response in the relative APTT value is observed as the dose of RB006 is escalated from 15 mg to 60 mg. The half-life of the pharmacodynamic activity of RB006 as assessed by the APTT assay appears to be at least 12 to 18 hrs, as this is the time required for the mean relative APTT for subjects treated with 60 mg RB006 to decay to the maximum relative APTT observed in subjects treated with 30 mg RB006.

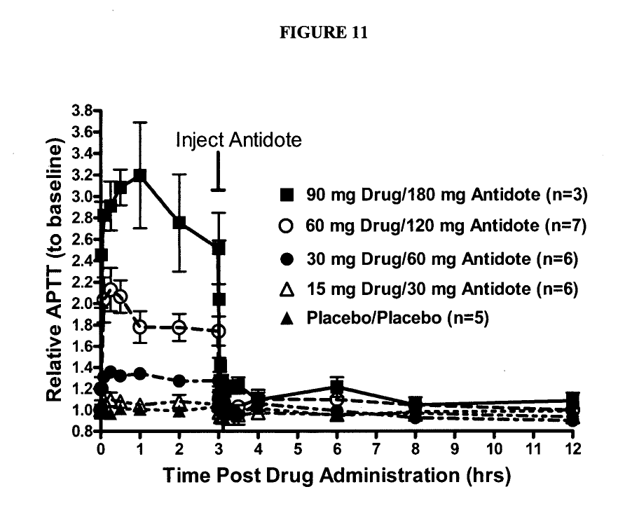

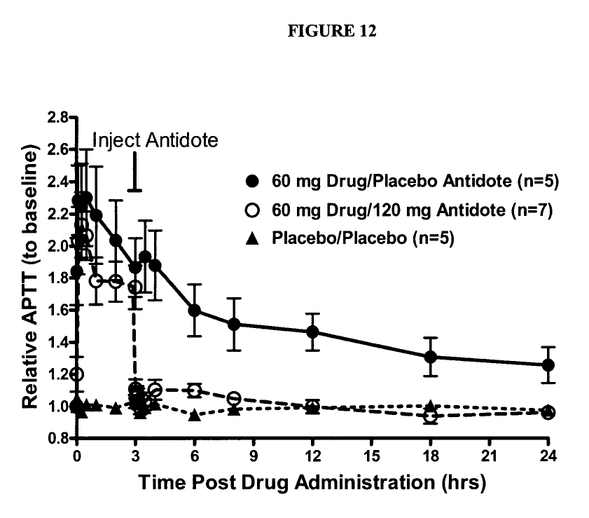

Subjects were administered RB006 or 0.9% normal saline (placebo) as an intravenous bolus injection at time zero, and then either RB007 or placebo, as an intravenous bolus injection at 3 hours post RB006 administration. The anticoagulant effect of the RB006 treatment was assessed over time by measurement of the plasma APTT (FIG. 11). APTT values for each treatment group are expressed as the mean±SEM of the Relative APTT. The Relative APTT is the APTT value for an individual subject at a given sample time divided by the pre-RB006 administration baseline APTT value for that subject. A clear dose-response in the relative APTT value is observed as the dose of RB006 is escalated from 15 mg to 90 mg. Administration of RB007 resulted in a complete, rapid (within 5 minutes) and durable neutralization of the pharmacologic activity of RB006 as evidenced by the return of the Relative APTT to baseline values following RB007 administration.

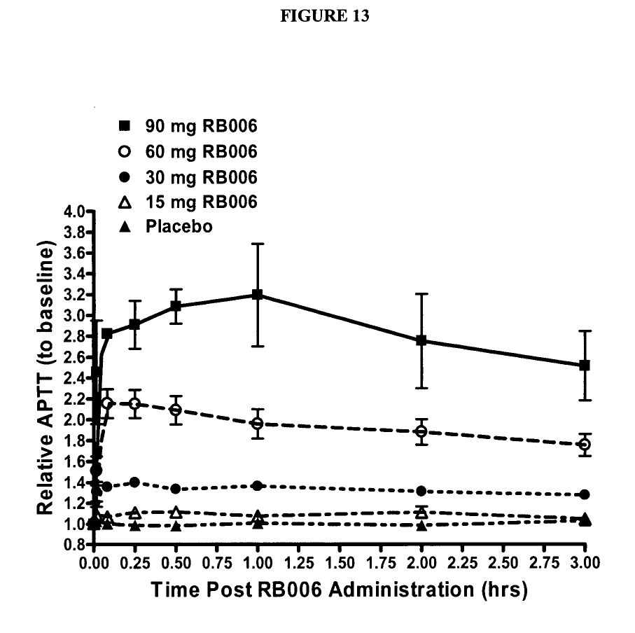

Treatments as described in above FIGS. 10 and 11. Comparison of the pharmacodynamic response in subjects treated with 60 mg RB006 followed by treatment with RB007 versus placebo at 3 hours demonstrates the rapid and durable neutralization activity of RB007 (FIG. 12). Administration of RB007 effectively eliminates exposure of the subjects to further anticoagulation, as visualized by the comparison of the area under the APTT response curve between 3 and 24 hours with and without RB007 administration.

The ability to administer the REG1 coagulation system in bolus IV injections without resultant complement activation in primates is surprising, given the association of complement activation, and thus toxicity, observed with the administration previously observed with such bolus injection administrations of other types of oligonucleotide molecules. See, for example, Galbraith et al. (1994) “Complement activation and hemodynamic changes following intravenous administration of phosphorothioate oligonucleotides in the monkey,” Antisense Research and Development 4:201-206; and Levin, A. A., Monteith, D. K., Leeds, J. M., Nicklin, P. L., Geary, R. S., Butler, M., Templin, M. V., and Henry, S. P. (1998). Toxicity of oligonucleotide therapeutic agents, In Handbook of Experimental Pharmacology, G. V. R. e. a. Born, ed. (Berlin: Springer-Verlag), pp. 169-215.

### Strategic Analysis of Dosing Parameters

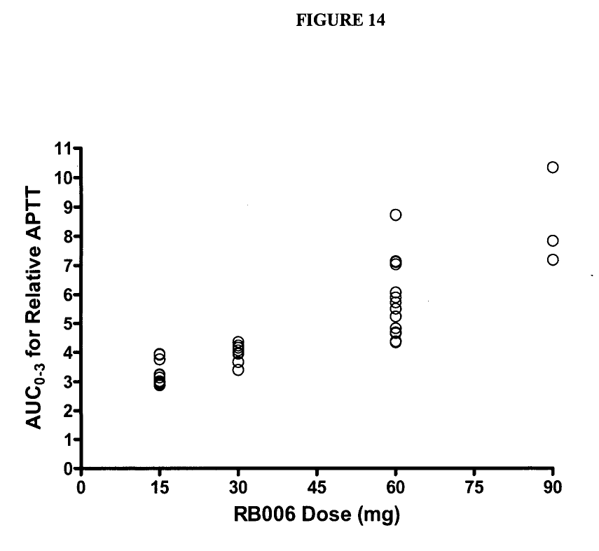

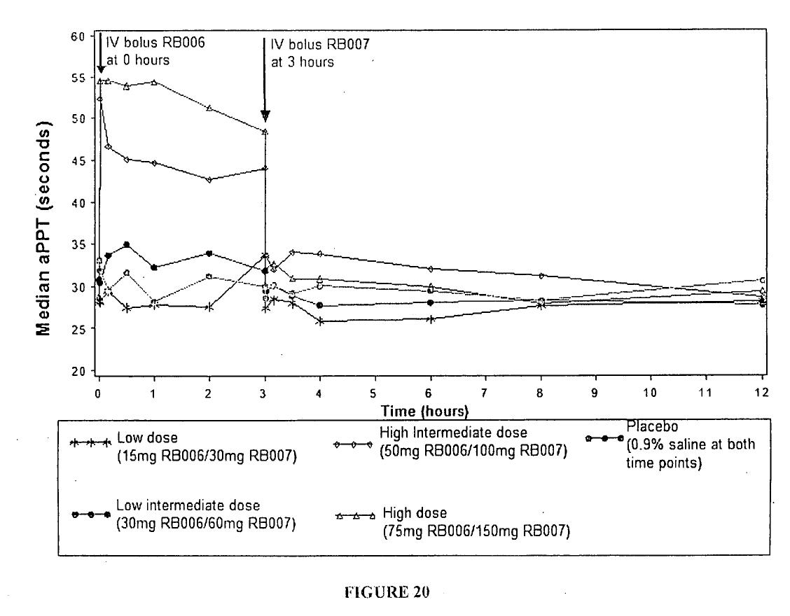

FIG. 13 shows a more detailed analysis of the relative increase in APTT over baseline from 0-3 hrs for all subjects who received RB006. Consistent with data from monkey trials, the level of APTT reaches a maximum and plateaus for several hours. The data were analyzed by assessing the area under the curve of the relative APTT as compared to baseline measured for the first three hours after treatment. FIG. 19 shows how the RB006 response relates to % FIX inhibition. This data shows that >99% FIX activity can be inhibited in a step-wise fashion using the anticoagulant.

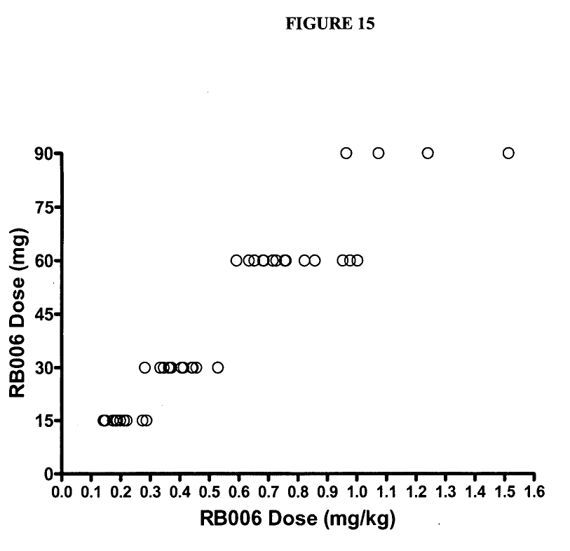

FIG. 14 shows the AUC 0-3 for each subject organized by RB006 dose level (15, 30, 60 or 90 mg). Because the relative effect is being measured over 3 hrs, a value of “3” represents no response, a value of 6 indicates an average 2 fold increase over baseline, etc.

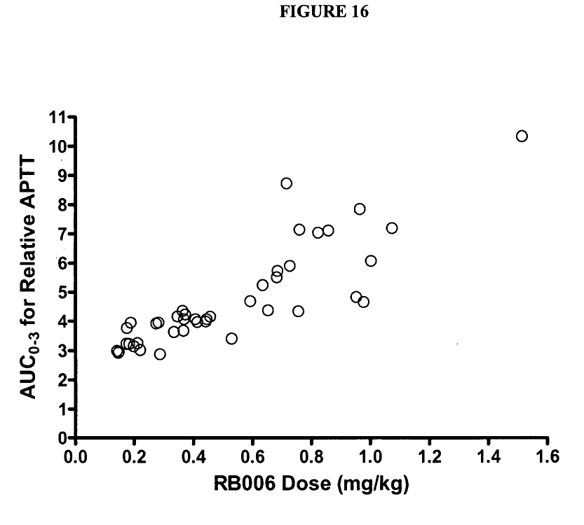

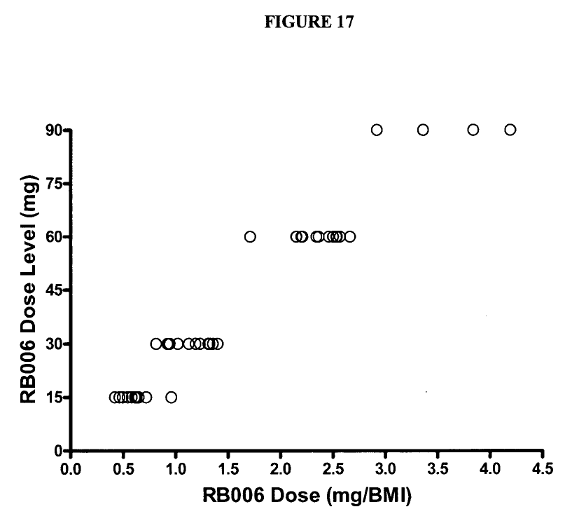

FIG. 15 shows the weight-adjusted dose of RB006 as a function of RB006 dose level. FIG. 16 depicts the relationship between the pharmacodynamic effect of RB006 (AUC 0-3) and the “weight adjusted” dose of RB006. The weight adjusted dose ranges from 0.2 mg/kg to 1.6 mg/kg, with a range of AUC0-3 from approximately 3 to 10 units. The graph shows that there is a clear relationship between response and the weight adjusted dose, with fairly low intersubject variability for an anticoagulant.

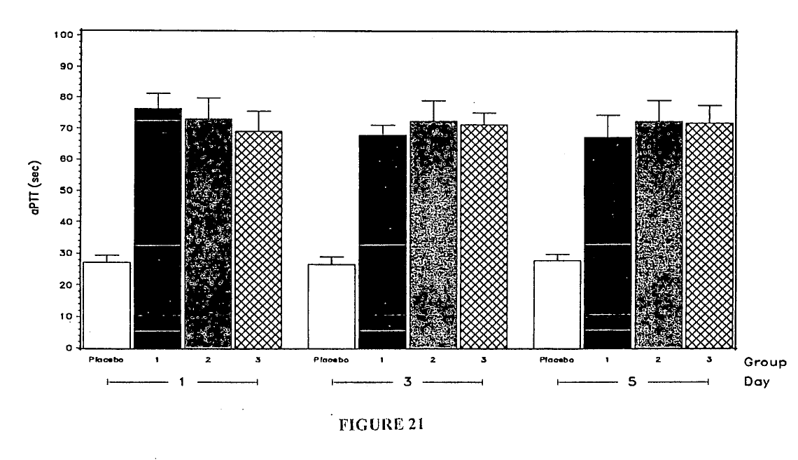

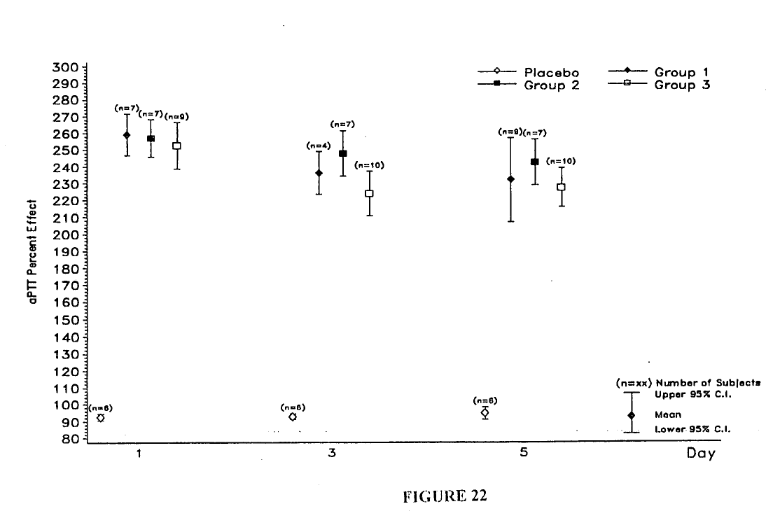

As seen in FIGS. 20 and 21, there is a clear relationship between body mass index (BMI) of enrolled subjects versus RB006 dose level. A BMI of 19-25 is normal, 25-30 is overweight and >30 is obese. Subjects in the study ranged from a BMI of approximately 16 to a BMI of over 35. Body Mass Index (BMI) is a number calculated from a person's weight and height. BMI is a reliable indicator of body fatness for people. BMI does not measure body fat directly, but research has shown that BMI correlates to direct measures of body fat, such as underwater weighing and dual energy x-ray absorptiometry (DXA). BMI can be considered an alternative for direct measures of body fat. BMI is calculated the same way for both adults and children. The calculation is based on the following formulas:

| Measurement | |
|---|---|
| units | Formula and calculation |
| Kilograms and | Formula: weight (kg)/[height (m)]² |
| meters (or | Calculation: [weight (kg)/height (m)/height |
| centimeters) | (m)] |
| | With the metric system, the formula for BMI is |
| | weight in kilograms divided by height in meters |
| | squared. Since height is commonly measured in |
| | centimeters, divide height in centimeters by 100 |
| | to obtain height in meters. |
| Pounds and inches | Formula: weight (lb)/[height (in)]² × 703 |
| | Calculation: [weight (lb)/height (in)/height |
| | (in)] × 703 |
| | Calculate BMI by dividing weight in pounds (lbs) |
| | by height in inches (in) squared and multiplying |
| | by a conversion factor of 703. |

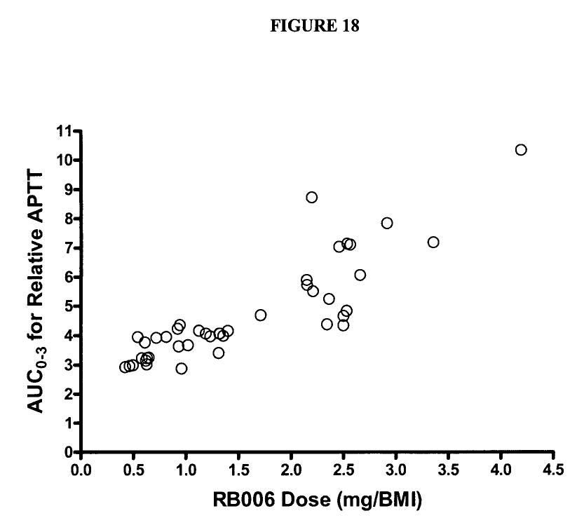

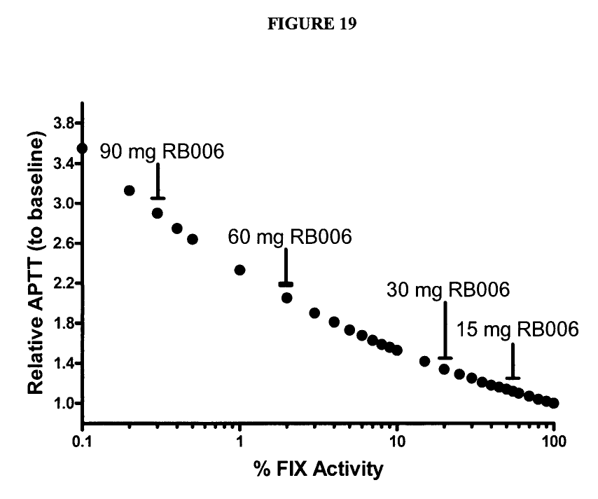

FIG. 17 shows the BMI adjusted dose of subjects treated with RB006 as a function of RB006 dose level. FIG. 18 depicts the relationship btw the AUC0-3 for RB006 versus BMI adjusted dose. Dosages ranged from 0.5 mg/BMI to approximately 4.5 mg/BMI. The range of AUC0-3 was between approximately 3 and 10 units. As can be seen in the graph, there is a clear relationship between pharmacodynamic parameters and the dosage adjusted for BMI. The relationship is even more pronounced than the weight adjusted dose relationship, with lower variability. The relationship of BMI to relative AUC0-3 indicates the drug is likely distributing mainly in the central body compartment, not to fat or related tissues. This distribution provides additional support for use of the REG1 system as an anticoagulant for parenteral administration.

Evaluation of the REG1 System in Patients with Stable CAD

Studies were conducted on 50 patients with stable coronary artery disease taking aspirin and/or clopidogrel. Patients were randomised to one of three groups (RB006 alone, RB006 followed by RB007, or placebo alone) across 4 dose levels of RB006 and RB007.

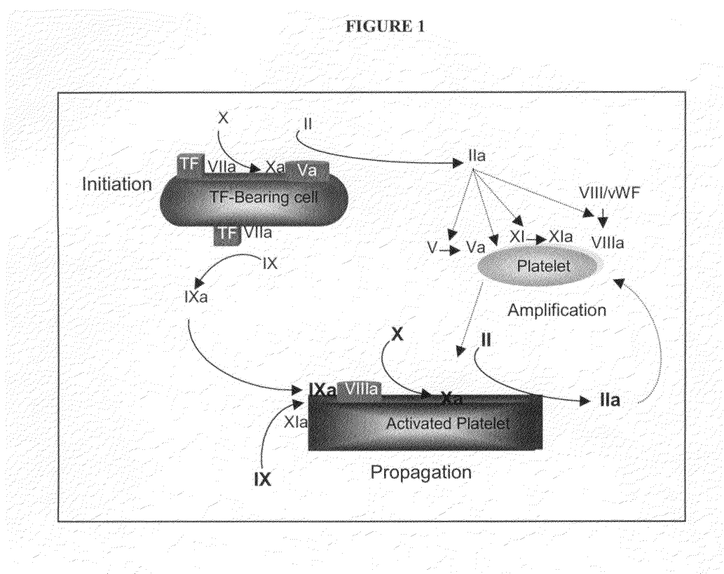

Baseline characteristics included a median age of 61 years (interquartile range (IQR) 56-68), 20% female, 80% prior percutaneous coronary intervention, and 34% prior coronary artery bypass grafting. The median aPTT 10 min after a single intravenous (IV) bolus of the low, low-intermediate, high intermediate and high dose of RB006 was 29.2 sec (IQR 28.1-29.8), 34.6 sec (IQR 30.9-40.0), 46.9 sec (IQR 40.3-51.1) and 52.2 sec (IQR 46.3-58.6), p<0.0001, (aPTT normal range 27-40 sec). RB007 reversed the aPTT to <10% above the upper limit of normal within a median of 1 min (IQR 1-2) (FIG. 1), with no rebound increase up to 7 days. Despite the use of dual anti-platelet therapy in 38% of subjects, there were no major bleeding or other serious adverse events.

FIG. 20 shows the results of a comparison of APTT response in four aptamer/antidote doses compared to placebo. Group 1 “low dose” was administered 15 mg RB006 at time 0 and 30 mg RB007 antidote at 3 hours in an IV bolus. Group 2 “low intermediate dose” was administered 30 mg RB006 at time 0 and 60 mg RB007 antidote at 3 hours in an IV bolus. Group 3 “high intermediate dose” was administered 50 mg RB006 at time 0 and 100 mg RB007 antidote at 3 hours in an IV bolus. Group 4 “high dose” was administered 75 mg RB006 at time 0 and 150 mg RB007 antidote at 3 hours in an IV bolus. At both 50 and 75 mg/kg RB006, a strong elevation in aPTT was seen, which was completely reversed upon administration of RB007 at 2× the aptamer concentration.

### Repeated Dosing of REG1 System

Studies were conducted on 38 patients in generally good health. Three treatment groups were identified: Group 1, in which subjects received a single dose of the aptamer (0.75 mg/kg RB006) on days 1, 3, and 5, followed by a fixed-dose of antidote (1.5 mg/kg RB007) one hour later and Groups 2 and 3, in which subjects received a single dose of aptamer RB006 (0.75 mg/kg) on days 1, 3, and 5, followed by varying single doses of RB007 administered one hour later. The dose titration for RB007 in subjects in Groups 2 and 3 is presented in Table A below.

TABLE A
Antidote (RB007) to Drug (RB006)
Dosing Ratio for Groups 2 and 3.

| | | |
|---|---|---|
| Day | Antidote:Drug Ratio | RB007 (mg/kg):RB006 (mg/kg) |

Group 2

| | | |
|---|---|---|
| 1 | 2:1 | 1.5:0.75 |
| 3 | 1:1 | 0.75:0.75 |
| 5 | 0.5:1 | 0.375:0.75 |

Group 3

| | | |
|---|---|---|
| 1 | 0.2:1 | 0.15:0.75 |
| 3 | 1:1 | 0.75:0.75 |
| 5 | TBD¹ | TBD:0.75 |

¹The antidote:drug ratio tested on Day 5 was between 0.1:1 and 1:1, and was based on the aPTT results from Days 1 and 3.
²Antidote dose was between 0.075 mg/kg and 0.75 mg/kg.

The dose of RB006 (0.75 mg/kg) was selected based on the body weight-adjusted response to RB006. On average, this weight-adjusted dose of RB006 elevated the subjects' APTT 2-fold. The RB006 aptamer, antidote and their respective placebos was each given as an injection over a period of one (1) minute. FIG. 21 shows the time-weighted APTT after RB006 (0.75 mg/kg) administration at days 1, 3 and 5 across different treatments of antidote.

FIG. 22 shows the percent effect on APTT of the administration of RB006 in the respective groups. An approximately 270% increase in APTT was seen after administration of 0.75 mg/kg aptamer in all three groups and did not differ significantly across the three treatment days.

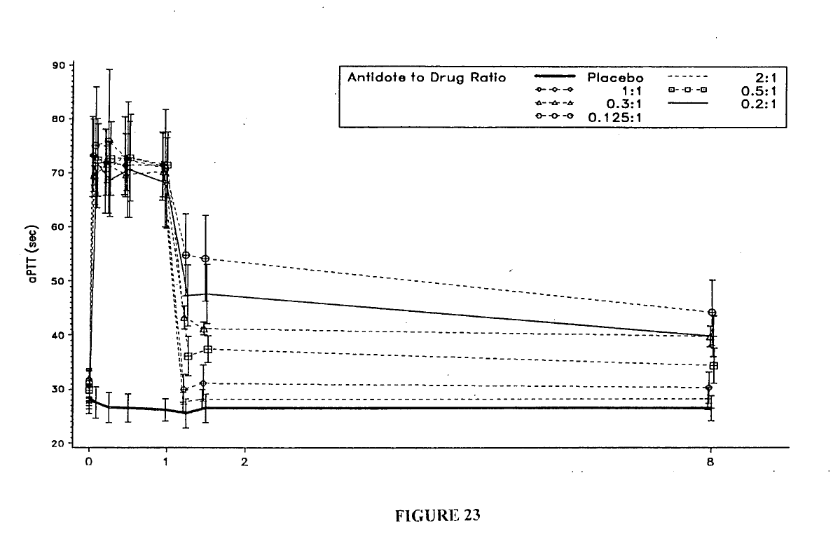

FIG. 23 shows the mean APTT in groups administered RB006 (0.75 mg/kg) and RB007 at various ratios compared to RB006. RB006 was administered at time 0 and RB007 at the listed ratios administered at one hour. As can be seen in the graph, RB007 reversed the anti-coagulant dose of antidote to aptamer. Furthermore, as can be seen in FIG. 23, the reversal effect of RB007 at each ratio tested was relatively stable over time, with a gradual reduction in RB006 pharmacodynamic activity over time as expected for this compound.

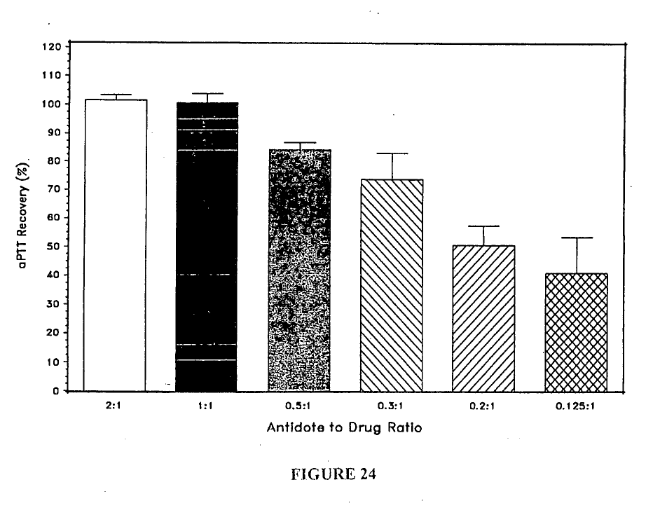

FIG. 24 shows the percent recovery in time weighted APTT in groups administered RB006 (0.75 mg/kg) and RB007 at various ratios compared to RB006. RB006 was administered at time 0 and RB007 at the listed ratios administered at one hour. At the lowest ratio tested, 0.125:1, RB007 reversed the effect of RB006 approximately 40%. At 0.2:1, RB007 reversed the effect of RB006 approximately 50%. At 0.3:1, RB007 reversed the effect of RB006 approximately 75%. At 0.5:1, RB007 reversed the effect of RB006 approximately 85%. And at higher ratios, of either 1:1 or 2:1, RB007 effectively completely reversed the effect of RB006.

1. A method of administration of an aptamer comprising:
a. measuring the body mass index (BMI) of a host;
b. identifying a desired pharmacodynamic response; and
c. administering to the host a dose of an aptamer to achieve a desired pharmacodynamic response based on a comparison of the dose per BMI to pharmacodynamic response.

2. The method of claim 1 further comprising administering a dose of an antidote to the aptamer to the host where the dose of antidote is based on the known dose of aptamer previously administered, and the antidote:aptamer ratio is based on a desired reduction in aptamer activity.

3. The method of claim 1 wherein the desired pharmacodynamic response is a maximal level of anti-coagulation.

4. The method of claim 3 wherein the aptamer is administered at a dose of 4 mg/BMI or greater.

5. The method of claim 1 wherein the desired pharmacodynamic response is a level of anticoagulation of about 75% maximal.

6. The method of claim 5 wherein the aptamer is administered at a dose of about between 3.0-4.0 mg/BMI.

7. The method of claim 1 wherein the desired pharmacodynamic response is a level of anticoagulation of about 50% maximal.

8. The method of claim 7 wherein the aptamer is administered at a dose of about between 2.0-3.0 mg/BMI.

9. The method of claim 1 wherein the dose of anticoagulant is between 0.1 and 10 mg/BMI.

10. The method of claim 1 wherein the dose of anticoagulant is about 5 mg/BMI.

11. A method of administration of an aptamer comprising:
a. measuring the weight in kg of a host;
b. identifying a desired pharmacodynamic response;
c. administering to the host a dose of an aptamer to achieve a desired pharmacodynamic response based on a comparison of the dose per kg to pharmacodynamic response; and,
d. administering a dose of an antidote to the aptamer to the host where the dose of antidote is provided based only on a ratio with aptamer

12. The method of claim 11 further comprising administering a dose of an antidote to the aptamer to the host where the dose of antidote is based on the known dose of aptamer previously administered, and the antidote:aptamer ratio is based on a desired reduction in aptamer activity.

13. The method of claim 11 wherein the desired pharmacodynamic response is maximal level of anti-coagulation.

14. The method of claim 13 wherein the dose of anticoagulant is about 1.4 mg/kg or greater.

15. The method of claim 11 wherein the desired pharmacodynamic response is a level of anticoagulation of about 75% maximal.

16. The method of claim 15 wherein the dose of anticoagulant is about between 1.0 mg/kg.

17. The method of claim 11 wherein the desired pharmacodynamic response is a level of anticoagulation of about 50% maximal.

18. The method of claim 17 wherein the dose of anticoagulant is about 0.6-0.8 mg/kg.

19. The method of claim 11 wherein the dose of anticoagulant is between 0.1 and 2 mg/kg.

20. The method of claim 11 wherein the dose of anticoagulant is between 5 and 10 mg/kg.

21. The method of claim 1 or 11 wherein the antidote is an oligonucleotide antidote.

22. The method of claim 1 or 11 wherein the aptamer comprises SEQ ID NO 1.

23. The method of claim 1 or 11 wherein the pharmacodynamic response is measured in a coagulation assay.

24. The method of claim 1 or 11 wherein the aptamer is administered in an IV bolus delivery.

25. The method of claim 1 or 11 wherein the aptamer is administered by subcutaneous injection.

26. The method of claim 2 or 12 wherein aptamer and antidote are administered at a ratio of 1:1.

27. The method of claim 2 or 12 wherein aptamer and antidote are administered at a ratio of at least 2:1.

28. The method of claim 2 or 12 wherein aptamer and antidote are administered at a ratio of 0.5:1 or less.

29. The method of claim 2 or 12 wherein aptamer activity is reversed by less than 90%.

30. The method of claim 2 or 12 wherein aptamer activity is reversed by about 50%.
